(19) United States
(12) Patent Application Publication
(10) Pub. No.: US 2022/0349013 A1
(43) Pub. Date: Nov. 3, 2022

(54) DETECTION AND TREATMENT OF RESIDUAL DISEASE USING CIRCULATING TUMOR DNA ANALYSIS

(71) Applicant: THE TRANSLATIONAL GENOMICS RESEARCH INSTITUTE, Phoenix, AZ (US)

(72) Inventors: Muhammed MURTAZA, Phoenix, AZ (US); Bradon MCDONALD, Phoenix, AZ (US); Tania CONTENTE-CUOMO, Phoenix, AZ (US); Ahuva ODENHEIMER, Phoenix, AZ (US)

(21) Appl. No.: 17/621,528

(22) PCT Filed: Jun. 25, 2020
(86) PCT No.: PCT/US2020/039701

§ 371 Date: Dec. 21, 2021

Related U.S. Application Data
(60) Provisional application No. 62/866,543, filed on Jun. 25, 2019.

(51) Int. Cl. C12Q 1/6886; G16B 20/20

(52) U.S. Cl. CPC C12Q 1/6886; G16B 20/20; C12Q 1/6858

(57) ABSTRACT

The present disclosure provides a method of treating a cancer in a patient who has undergone a first anti-cancer therapy. Also provided is a method for monitoring treatment response and minimum residual disease in a neoadjuvantly treated cancer patient. Methods for primer design are also disclosed. The present disclosure provides several tools for increasing the sensitivity and analytical precision of the disclosed methods for monitoring ctDNA.

### CROSS-REFERENCE TO RELATED APPLICATIONS

This application claims priority to U.S. Provisional Application No. 62/866,543, filed Jun. 25, 2019, the contents of which are incorporated herein by reference in their entirety.

### INCORPORATION-BY-REFERENCE OF MATERIAL ELECTRONICALLY FILED

Incorporated by reference in its entirety herein is a computer-readable nucleotide/amino acid sequence listing submitted concurrently herewith and identified as follows: One 5,172 byte ASCII (text) file named “Seq_List” created on Jun. 22, 2020.

### TECHNICAL FIELD

This application relates to methods of treating a cancer in a patient who has undergone a first anti-cancer therapy as well as monitoring treatment response and minimum residual disease (MRD) in a neoadjuvantly treated cancer patient.

### BACKGROUND

To maximize the rate of cure, cancer patients with non-metastatic disease are often treated with multiple modalities including pre-operative systemic and radiation therapy, surgery and post-operative therapy. However, for some patients, this results in overtreatment and adverse effects when they could have been cured with less intensive treatment and the benefit of each consecutive modality of therapy is not certain (1). A treatment monitoring biomarker that can accurately distinguish residual disease from disease eradication could enable a new paradigm for individualized management of localized cancers, but this has remained elusive because current diagnostics have inadequate sensitivity. In breast cancer, ˜30% patients treated with neoadjuvant therapy achieve pathological Complete Response (pathCR) with no histological evidence of invasive tumor in the resected breast tissue and lymph nodes (2). pathCR during neoadjuvant therapy is associated with excellent long-term clinical outcomes. Ten year relapse free survival rates are 95%, 86% and 83% in patients with Human Epidermal growth factor Receptor 2-positive (HER2+), Triple-Negative (TNBC) and Estrogen Receptor-positive, Human Epidermal growth factor Receptor 2-negative (ER+HER2−) breast cancer respectively (3). In these patients, it is uncertain whether surgery provides any further therapeutic benefit, although it adds diagnostic value by confirming pathCR. An alternative diagnostic test to accurately detect residual disease could guide choice and planning of local treatment options such as the extent of surgical resection or the use of radiation therapy (4, 5).

Recent advances in circulating tumor DNA (ctDNA) analysis have shown promise in monitoring non-metastatic cancer patients but these have primarily focused on recurrence monitoring and lack accuracy for residual disease detection during treatment (6-9). In particular, detection of ctDNA after completion of neoadjuvant therapy has been challenging in patients with breast and rectal cancer, even when residual disease is observed at the time of surgery. Recent studies have found ctDNA becomes undetectable in more than 90% of patients during neoadjuvant therapy due to limited assay sensitivity (10). As a result, no association has been observed between ctDNA detection from blood and residual disease after completion of neoadjuvant therapy (11,12). Detection of low levels of ctDNA in non-metastatic cancer patients is impeded by limited blood volumes accessible in a clinical environment and low concentrations of total cell-free DNA (hereon cfDNA). Unlike in metastatic cancer patients where cfDNA concentrations are much higher, a 10 mL blood tube (4 mL plasma) from early stage cancer patients typically yields 20 ng cfDNA (˜6000 haploid genome copies). In addition, ctDNA levels in early and locally advanced cancer patients are lower compared to metastatic cancer patients. For example, prior to treatment, median ctDNA levels in triple negative breast cancer (TNBC) have been reported at 12.5% in metastatic cancer patients and at 0.68% in non-metastatic cancer patients (almost 20 fold lower) (12, 13). During and after completion of treatment, any ctDNA signal from residual disease is expected to be at even lower levels. As a result, sensitivity and analytical precision of ctDNA tests for residual disease are often limited due to stochastic sampling variation (FIG. 1A). Thus, there is a need to develop methods of monitoring ctDNA with increased sensitivity and analytical precision to guide the treatment of cancer patients and, particularly, those suffering from early and locally advanced cancer where ctDNA levels are relatively low.

### SUMMARY

The present disclosure provides several tools for increasing the sensitivity and analytical precision of the disclosed methods for monitoring ctDNA. Sampling variation can be overcome by increasing the volume of blood obtained at each time point to increase the amount of total plasma DNA analyzed, by improving the rate of conversion of DNA into sequencing-ready molecules and by simultaneously analyzing multiple patient-specific somatic founder mutations. Founder mutations are present in all cancer cells and therefore, each is equally informative of tumor-derived DNA in blood (14). To leverage these principles and enable residual disease detection, we have developed a personalized approach for tumor-guided ctDNA detection and quantification called TARgeted DIgital Sequencing (TARDIS). Here, we describe development and analytical performance of TARDIS using dozens of replicates of reference materials with tumor fractions as low as 3 in 10⁵ and we demonstrate clinical performance of ctDNA detection and quantification in patients with early and locally advanced breast cancer, prior to and after completion of neoadjuvant systemic therapy.

More specifically, a method of treating a cancer in a patient who has undergone a first anti-cancer therapy is disclosed herein. The method typically comprises the following steps: a) obtaining double-stranded cell-free DNA (cfDNA) from a blood sample from the patient, e.g., 1 to 50 nanograms (ng) of double-stranded cfDNA; b) linearly amplifying the cfDNA with target-specific primers to generate single-stranded DNA amplicons, wherein the target-specific primers are generated from a genetic profile of the patient; c) ligating an adapter oligonucleotide to the 3′-ends of the single-stranded DNA amplicons, d) performing multiplexed, exponential amplification with target-specific primers and nested primers on the single-stranded DNA amplicons to produce parent polynucleotides; e) amplifying the parent polynucleotides to produce progeny polynucleotides with associated sample barcodes; f) sequencing a portion of the progeny polynucleotides to produce sequencing reads of the progeny polynucleotides with associated sample barcodes; f) aligning mappable portions of the sequencing reads to a human reference genome; g) grouping a plurality of the sequencing reads into clusters based on the sequence information of the sample barcodes and the beginning and end base positions of the mapped portion of the progeny polynucleotides; h) detecting, from among a plurality of the clusters, the presence or absence of one or more somatic genetic variants characteristic of the cancer, wherein the presence of the one or more somatic genetic variants or the aggregate quantification of somatic genetic variants surpassing a threshold, indicates cancer persistence and/or recurrence; and i) administering a second anti-cancer therapy to the patient once cancer recurrence is detected.

In another aspects, the disclosure is directed to a method of monitoring treatment response and minimum residual disease (MRD) in a neoadjuvantly treated cancer patient. The method comprises the steps of: a) obtaining double-stranded cfDNA from a blood sample from the patient, e.g., 1 to 50 ng; b) linearly amplifying the cfDNA with target-specific primers to generate single-stranded DNA amplicons, wherein the target-specific primers are generated from a genetic profile of the patient; c) ligating an adapter oligonucleotide to the 3′-ends of the single-stranded DNA amplicons, d) performing multiplexed, exponential amplification with target-specific primers and nested primers on the single-stranded DNA amplicons to produce parent polynucleotides; e) amplifying the parent polynucleotides to produce progeny polynucleotides with associated sample barcodes; f) sequencing a portion of the progeny polynucleotides to produce sequencing reads of the progeny polynucleotides with associated sample barcodes; f) aligning mappable portions of the sequencing reads to a human reference genome; g) grouping a plurality of the sequencing reads into clusters based on the sequence information of the sample barcodes and the beginning and end base positions of the mapped portion of the progeny polynucleotides; and h) detecting, from among a plurality of the clusters, the presence or absence of one or more somatic genetic variants characteristic of the cancer, wherein the presence of the one or more somatic genetic variants or the aggregate quantification of somatic genetic variants surpassing a threshold indicates cancer recurrence and/or a need for adjustment in cancer treatment.

In certain advantageous embodiments, the method also includes generating a report that includes a cell-free tumor mutation profile of the patient based on the detection of the presence or absence of the one or more somatic genetic variants, which may include a treatment recommendation based on the cell-free mutation profile. The genetic profile may comprise patient-specific putative founder mutations identified with whole genome or whole exome sequencing of tumor biopsy DNA and germline DNA from the patient.

In particular embodiments, the patient has early stage cancer and the blood sample comprises less than 5 ng cfDNA/mL, less than 4 ng cfDNA/mL, less than 3 ng cfDNA/mL, less than 2 ng cfDNA/mL, or less than 1 ng cfDNA/mL.

The disclosure also provides useful primers. For examples, primers comprising SEQ ID NO: 2 and SEQ ID NO: 3, for performing multiplexed, exponential amplification; primers comprising SEQ ID NO: 4 and SEQ ID NO: 5 for associating sample barcodes with progeny nucleotides; primers comprising SEQ ID NO: 6 and SEQ ID NO: 7 useful in sequencing of progeny nucleotides using next generation sequencing.

Other useful primers disclosed herein include the following forward and reverse primers comprising: SEQ ID NO: 8 and SEQ ID NO: 9; SEQ ID NO: 10 and SEQ ID NO: 11; SEQ ID NO: 12 and SEQ ID NO: 13; SEQ ID NO: 14 and SEQ ID NO: 15; SEQ ID NO: 16 and SEQ ID NO: 17; SEQ ID NO: 18 and SEQ ID NO: 19; SEQ ID NO: 20 and SEQ ID NO: 21; and/or SEQ ID NO: 22 and SEQ ID NO: 23.

In a particular advantageous embodiment, the target-specific primers simultaneously amplify target regions comprising at least 10, at least 50, or at least 100 mutations in the cfDNA and/or the amplify target regions comprise a genomic sequence selected from the group consisting of: AKT, GNAQ, GNA11, IDH1, TP53, KRAS, PDGFRA, PIK3CA, APC, EGFR, BRAF, MET, MYC, and RET.

Certain non-limiting of examples of adapter oligonucleotides useful in the disclosed methods include adapter oligonucleotides comprising: a stem-loop intramolecular nucleotide base pairing; a hydroxyl group at the 3′-end; a phosphate at the 5′-end; a random region complementary to the nucleic acid sequence; and a random region in the loop comprising a unique molecular identifier (UMI). In a specific example the adapter oligonucleotide comprises SEQ ID NO: 1.

In particular embodiment, the methods further provide the step of differentiating true low-abundance somatic genetic variants from nucleotide misincorporations that occur during amplification or from nucleotide misreads that occur during sequencing by: grouping sequencing reads based on fragment size and UMI into read families; requiring consensus among all sequencing reads in a read family; and requiring that a true low-abundance somatic genetic variant be supported by at least two independent read families of different fragment size. In specific nonlimiting embodiments: the product of an allele fraction for a somatic genomic variant with the known level of input cfDNA amount in genomic equivalents is equivalent to at least 0.5 DNA fragments; the read families covering each targeted genomic locus are sorted by their size (number of members), such that read families with the most members up to 5-fold of known level of input cfDNA in genomic equivalents are considered for detection of somatic genomic variants; and/or the cut-off for detection of somatic genomic variants is less than 5-fold or greater than 5-fold.

In yet another aspect, the methods disclosed further comprise calculating a probability of observing each somatic genetic variant based on a background distribution of mutations in the cfDNA, applying multiple testing correction using the Bonferroni approach, and requiring a corrected p-value of <0.05 to distinguish true low-abundance somatic genetic variants from nucleotide misincorporations that occur during amplification or from nucleotide misreads that occur during sequencing, and to determine whether a sample is positive for tumor contribution in cfDNA. In another aspect of the methods, mixed read families (RFs) containing multiple members that disagree on nucleotide identity at a target genomic locus, provide an assessment of error propensity at the locus. A heuristic or probabilistic approach can used to differentiate low-abundance somatic genetic variants from nucleotide misincorporations or sequencing errors.

In a specific non-limiting embodiment, the method further includes calculating the background distribution of mutations in cfDNA specific to the sequenced biological sample, wherein the mutation background distribution is calculated using data from adjacent and/or non-adjacent genomic loci, not expected to be mutated. In a particular example, the background distribution of mutations in cfDNA is calculated using data from an unrelated set of biological samples not expected to be mutated at the targeted genomic locus or at unrelated genomic loci.

A positive detection of tumor contribution in cfDNA is supported by at least one somatic genomic alteration supported by at least two read families of independent size having one somatic genomic alteration.

The disclosure is also directed to a method of designing a primer design. The method typically comprises: a) identifying multiple founder mutations by analyzing tumor tissue using next generation sequencing to target for analysis in cfDNA; b) designing primers within preset thresholds for GC content, multiple temperature and length such that multiple pairs of primer (two primers in each pair) are identified for each target, wherein, both primers in a pair are on the same strand and on either side of the targeted genomic locus, and wherein both primers are within 300 bp of the targeted locus; c) evaluating off-target annealing for primers across the genome using informatic approaches; d) sorting primers by distance of 3′end of the primer to the target, to minimize this distant as much as possible; e) removing redundant primers with different lengths that share the same 3′ end; f) evaluating pairwise cross amplification between primers using informatic approaches, such as, in-silico PCR; g) removing primers that cross-amplify using a network-based method such that each primer is represented by a node and its interactions with other primers are represented by edges, removing nodes with the highest number of edges, such that only nodes with zero edges remain; h) evaluating primer performance by preparing TARDIS sequencing libraries using a set of control DNA samples; and i) removing a primer from the primer set based on the primer crossing a threshold comprising: the median proportion of soft masked reads across replicates is greater than 0.5; the max proportion of soft masked reads in any replicate is greater than 0.75; the median read proportion across replicates for the primer is greater than 4*(1/total primer count); the max proportion in any replicate is greater than 0.75; the median proportion of reads in the most abundant size is greater than 0.25; the max proportion in the most abundant size is greater than 0.5 in any replicate; or the median number of molecule sizes is less than 20.

In particular embodiments of the primer design method, the edges are represented by primer interactions determined using in silico PCR or the edges are represented by primer interactions determined using matching of the last 6 nucleotides in each primer with each other. Preferably, the number of nucleotides matched is less than or greater than 6. In one nonlimiting example, the edges are represented by primer interactions determined using matching of the last 6 nucleotides in each primer with 300 bp region around all other targeted loci, e.g., the number of nucleotides matched is less than or greater than 6, the number of regions around each target is less than or greater than 300 bp, or both.

### BRIEF DESCRIPTION OF THE DRAWINGS

FIGS. 1A-1D depict the development of a multiplexed assay for personalized ctDNA detection and monitoring. FIG. 1A depicts a graph demonstrating that based on binomial sampling, maximum theoretical sensitivity for detection of ctDNA at 0.001% tumor fraction is limited if only 2-4 mutations are assayed but can be improved with higher input of plasma DNA and increasing number of patient- specific mutations. FIG. 1B depicts a schematic showing how TARDIS identifies patient-specific putative founder mutations using exome sequencing of tumor biopsies and tracks multiple mutations simultaneously in plasma to monitor treatment response and to detect MRD. FIG. 1C depicts how TARDIS relies on a novel approach for preparing sequencing libraries that includes linear pre-amplification to improve molecular conversion, single-stranded DNA ligation using hairpin oligonucleotides to allow error suppression using template fragment sizes and unique molecular identifiers (UMIs) and multiplexed PCR to enrich targeted genomic loci. FIG. 1D depicts a schematic representation of error suppression using TARDIS. TARDIS uses UMIs and fragment sizes to group sequencing reads into read families (RFs). We exclude PCR errors by requiring consensus of all RF members and polymerase errors introduced during linear pre-amplification by requiring support by at least 2 RFs. Additional description of error suppression strategies is provided herein.

FIGS. 2A-2F depict the analytical performance of TARDIS in reference samples. FIG. 2A depicts mutation-level sensitivity and specificity across 93 reference samples and 8 mutations, requiring each mutation is supported by ≥2 RFs and an AF consistent with ≥0.5 mutant molecules. FIG. 2B depicts sample-level sensitivity and specificity, requiring ≥2 RFs contributed by one mutation with multiple sizes or >1 mutations, each with an AF consistent with ≥0.5 mutant molecules. FIG. 2C depicts a comparison of variant AFs observed using TARDIS (mean for each variant across all replicates at the same mutation level, 48 data points) with known variant AFs measured using ddPCR. Gray line is linear fit. FIG. 2D depicts a comparison of sample AFs observed using TARDIS (mean for all 8 mutations assayed in each replicate sample, 77 data points) with known sample AFs (mean of known variant AFs). Gray line is linear fit to the mean at each AF level. FIG. 2E depicts the CVs of variant AFs decreased with increasing number of mutant molecules per mutation. CVs calculated across 7-16 replicates at each mutation level for each of 8 mutations (48 data points). FIG. 2F depicts the CVs of sample-level AFs were lower than those for individual mutations, demonstrating the advantage of leveraging multiple mutations for ctDNA quantification. CVs calculated across 7-16 replicates for sample-level means across 6 mutation levels.

FIGS. 3A-3D depict an evaluation of analytical performance in reference samples at 3 in 10⁵ tumor fractions. FIG. 3A depicts the variant-level sensitivity and specificity across 56 reference samples and 16 mutations, requiring each mutation is supported by ≥2 RFs and an AF consistent with ≥0.5 mutant molecules. 22 mutations were analyzed in this experiment. However, 6 mutations were inferred to contribute biological background as these were recurrently observed in the wild-type DNA sample sourced from immortalized cell lines. These included known hotspot variants in TP53 (n=4/4 targets), APC (n=1/2 targets) and GNAS (n=1/1 targets). These mutations were dropped from further analysis. FIG. 3B depicts the sample-level sensitivity and specificity, requiring ≥2 RFs contributed by one mutation with multiple sizes or >1 mutations, each with an AF consistent with ≥0.5 mutant molecules. Although a mutation with 2 RFs was observed in 1 wild-type sample, this mutation was supported by a single size and at the sample-level, ctDNA was determined to be undetectable (see Methods for ctDNA detection criteria in the Examples). FIG. 3C depicts the accuracy evaluated by comparison of sample AFs observed using TARDIS (mean for all 16 mutations assayed in each replicate sample) with known sample AFs (mean of known variant AFs measured using digital PCR). Line is linear fit to the mean at each AF level. FIG. 3D depicts the precision evaluated using CVs of sample-level AFs, calculated across 8-32 replicates for sample-level means.

FIGS. 4A-4E depict a ctDNA analysis in patients with early and locally advanced breast cancer before treatment and after completion of neoadjuvant therapy. FIG. 4A depicts the clinical characteristics of the cohort. FIG. 4B depicts a summary of results, TNM staging and ctDNA detection before treatment and after neoadjuvant therapy. Pathological TNM staging was performed after surgery and completion of NAT. Number in each box indicates T or N stage, is: in situ, mi: microinvasive disease. FIG. 4C depicts ctDNA levels at baseline. FIG. 4D depicts ctDNA levels after completion of neoadjuvant therapy, grouped by clinical response to treatment (Residual Disease vs. pathological Complete Response). FIG. 4E depicts the change in pre- and post-treatment ctDNA levels in patients with residual disease and pathCR.

FIG. 5 depicts a Receiver Operating Characteristic Curve for predicting residual disease using ctDNA levels after completion of neoadjuvant therapy.

FIG. 6 depicts a comparison of raw and TARDIS-corrected background errors. Requiring consensus of all members of an RF, a minimum of 2 RFs with a ratio between variant RFs and mixed RFs<10, we observed background error rate drops significantly. Top panel shows raw error rates across 200 loci from each of 39 samples, for a total of 7,800 independent positions. Bottom panel shows TARDIS-corrected error rates.

FIG. 7 depicts a comparison of total cfDNA concentration between plasma samples from patients with early and locally advanced breast cancer (this study), healthy volunteers (data reported in Markus et al. 2018) and metastatic cholangiocarcinoma patients. All plasma DNA concentrations were measured using droplet digital PCR.

FIG. 8 depicts a comparison of individual mutation AFs and ctDNA tumor fractions in individual patients over multiple time points when available. Time point 1 is before treatment and time point 4 is after completion of neoadjuvant therapy for all patients. For one cohort (prefixed T), time points 2 and 3 were collected at 6 and 12 weeks during neoadjuvant therapy. Gray lines with circles show individual mutation AFs while black lines with crosses show tumor fractions calculated as described in the Examples in Methods (Data S4). Plasma DNA input levels varied across samples, limiting individual mutation sensitivity and precision.

### DETAILED DESCRIPTION

As used herein and in the appended claims, the singular forms “a,” “an,” and “the” include plural referents unless the context clearly dictates otherwise. For example, “a” or “an” means “at least one” or “one or more.” Thus, reference to “an antibody or antigen binding fragment thereof refers to one or more antibodies or antigen binding fragments thereof, and reference to “the method” includes reference to equivalent steps and methods disclosed herein and/or known to those skilled in the art, and so forth.

Throughout this disclosure, various aspects of the claimed patient matter are presented in a range format. It should be understood that the description in range format is merely for convenience and brevity and should not be construed as an inflexible limitation on the scope of the claimed patient matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed patient matter. The upper and lower limits of these smaller ranges may independently be included in the smaller ranges, and are also encompassed within the claimed patient matter, patient to any specifically excluded limit in the stated range. Where the stated range includes one or both of the limits, ranges excluding either or both of those included limits are also included in the claimed patient matter. This applies regardless of the breadth of the range.

A “patient” as used herein refers to an organism, or a part or component of the organism, to which the provided methods, apparatuses, and systems can be administered or applied. For example, the patient can be a mammal or a cell, a tissue, an organ, or a part of the mammal. Mammals include, but are not limited to, humans, and non-human animals, including farm animals, sport animals, rodents and pets.

The terms “nucleic acid,” “nucleotide,” “polynucleotide,” and “oligonucleotide” are used interchangeably. They refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

The term “biological sample” refers to a body sample from any animal, but preferably is from a mammal, more preferably from a human. Such samples include biological fluids such as serum, plasma, vitreous fluid, lymph fluid, synovial fluid, follicular fluid, seminal fluid, amniotic fluid, milk, whole blood, urine, cerebro-spinal fluid, saliva, sputum, tears, perspiration, mucus, and tissue culture medium, as well as tissue extracts such as homogenized tissue, and cellular extracts.

The terms “sequence variant” or “mutation” are used interchangeably and refer to any variation in a nucleic acid sequence including but not limited to single point-mutations, multiple point-mutations, insertions/deletions (indels), and single-nucleotide polymorphisms (SNPs). These terms are used interchangeably in this document, and it is understood that when reference is made to a method for evaluating one type of variant, it could be equally applied to evaluation of any other type of variant. The term “variant” can also be used to refer to a single molecule whose sequence deviates from a reference sequence, or a collection of molecules whose sequences all deviate from the reference sequence in the same way. Similarly, “variant” can refer to a single sequence (or read) that deviates from a reference sequence or a set of sequences that deviate from a reference sequence.

The terms “mutation-prone region” and “mutation hotspot” are used interchangeably, and refer to a sequence region of a nucleic acid obtained from a biological source that has a higher probability of being mutated than surrounding sequence regions within the same nucleic acid. In the case of tumor-derived DNA, mutation-prone regions can be found in certain cancer-related genes. The mutation-prone region can be of any length, but mutation-prone regions that are analyzed using the methods disclosed herein are less than 100 nucleotides long. A mutation can be found anywhere within a mutation-prone region.

The term “target region” refers to a region of a nucleic acid that is targeted for primer extension or PCR amplification by specific hybridization of complementary primers.

The terms “barcode”, “tag”, and “index” are used interchangeably and refer to a sequence of bases at certain positions within an oligonucleotide that is used to identify a nucleic acid molecule as belonging to a particular group. A barcode is often used to identify molecules belonging to a certain sample when molecules from several samples are combined for processing or sequencing in a multiplexed fashion. A barcode can be any length, but is usually between 6 and 12 bases long (need not be consecutive bases). Barcodes are usually artificial sequences that are chosen to produce a barcode set, such that each member of the set can be reliably distinguished from every other member of the set. Various strategies have been used to produce barcode sets. One strategy is to design each barcode so that it differs from every other barcode in the set at a minimum of 2 distinct positions.

Longitudinal analysis of circulating tumor DNA (ctDNA) has shown promise for monitoring treatment response. However, most current methods lack adequate sensitivity for residual disease detection during or after completion of treatment in non- metastatic cancer patients. To address this gap and to improve sensitivity for minute quantities of residual tumor DNA in plasma, we have developed TARgeted DIgital Sequencing (TARDIS) for multiplexed analysis of patient-specific cancer mutations. In reference samples, by simultaneously analyzing 8-16 known mutations, TARDIS achieved 91% and 53% sensitivity at mutant allele fractions (AF) of 3 in 104 and 3 in 105 respectively with 96% specificity, using input DNA equivalent to a single tube of blood. We successfully analyzed up to 115 mutations per patient in 80 plasma samples from 33 women with stage I-III breast cancer. Prior to treatment, TARDIS detected ctDNA in all patients with 0.11% median AF. After completion of neoadjuvant therapy, ctDNA levels were lower in patients who achieved pathological Complete Response (pathCR) compared to patients with residual disease (median AFs 0.003% and 0.017% respectively, p=0.0057, AUC=0.83). In addition, patients with pathCR showed a larger decrease in ctDNA levels during neoadjuvant therapy. These results demonstrate high accuracy for assessment of molecular response and residual disease during neoadjuvant therapy using ctDNA analysis. TARDIS has achieved up to 100-fold improvement in limit of ctDNA detection using clinically relevant blood volumes, demonstrating that personalized ctDNA tracking could enable individualized clinical management of cancer patients treated with curative intent.

In certain aspects, the disclosure provides a robust personalized ctDNA test, TARDIS, achieving high accuracy for residual disease after completion of neoadjuvant therapy.

In other aspects, the disclosure provides a method of treating a cancer in a patient who has undergone a first anti-cancer therapy. The method typically comprises: a) obtaining double-stranded cell-free DNA (cfDNA) from a blood sample from the patient, e.g., obtaining 1 to 50 nanograms (ng) of double-stranded cfDNA; b) linearly amplifying the cfDNA with target-specific primers to generate single-stranded DNA amplicons, wherein the target-specific primers are generated from a genetic profile of the patient; c) ligating an adapter oligonucleotide to the 3′-ends of the single-stranded DNA amplicons, d) performing multiplexed, exponential amplification with target-specific primers and nested primers on the single-stranded DNA amplicons to produce parent polynucleotides; e) amplifying the parent polynucleotides to produce progeny polynucleotides with associated sample barcodes; f) sequencing a portion of the progeny polynucleotides to produce sequencing reads of the progeny polynucleotides with associated sample barcodes; f) aligning mappable portions of the sequencing reads to a human reference genome; g) grouping a plurality of the sequencing reads into clusters based on the sequence information of the sample barcodes and the beginning and end base positions of the mapped portion of the progeny polynucleotides; and h) detecting, from among a plurality of the clusters, the presence or absence of one or more somatic genetic variants characteristic of the cancer, wherein the presence of the one or more somatic genetic variants indicates cancer recurrence; and i) administering a second anti-cancer therapy to the patient once cancer recurrence is detected.

In one aspect, the first anti-cancer therapy is different than the second anti-cancer therapy. In another aspect, the first anti-cancer therapy is the same as the second anti-cancer therapy.

### Sample Preparation

The methods of this disclosure may have a wide variety of uses in the manipulation, preparation, identification and/or quantification of cell free polynucleotides. Examples of polynucleotides include but are not limited to: DNA, RNA, amplicons, cDNA, dsDNA, ssDNA, plasmid DNA, cosmid DNA, high Molecular Weight (MW) DNA, chromosomal DNA, genomic DNA, viral DNA, bacterial DNA, mtDNA (mitochondrial DNA), mRNA, rRNA, tRNA, nRNA, siRNA, snRNA, snoRNA, scaRNA, microRNA, dsRNA, ribozyme, riboswitch and viral RNA (e.g., retroviral RNA).

Cell free polynucleotides may be derived from a variety of sources including human, mammal, non-human mammal, ape, monkey, chimpanzee, reptilian, amphibian, or avian, sources. Further, samples may be extracted from variety of animal fluids containing cell free sequences, including but not limited to blood, serum, plasma, vitreous, sputum, urine, tears, perspiration, saliva, semen, mucosal excretions, mucus, spinal fluid, amniotic fluid, lymph fluid and the like. Cell free polynucleotides may be fetal in origin (via fluid taken from a pregnant patient), or may be derived from tissue of the patient itself.

Isolation and extraction of cell free polynucleotides may be performed through collection of bodily fluids using a variety of techniques. In some cases, collection may comprise aspiration of a bodily fluid from a patient using a syringe. In other cases, collection may comprise pipetting or direct collection of fluid into a collecting vessel.

After collection of bodily fluid, cell free polynucleotides may be isolated and extracted using a variety of techniques known in the art. In some cases, cell free DNA may be isolated, extracted and prepared using commercially available kits such as the Qiagen Qiamp® Circulating Nucleic Acid Kit protocol. In other examples, Qiagen Qubit™ dsDNA HS Assay kit protocol, Agilent™ DNA 1000 kit, or TruSeq™ Sequencing Library Preparation; Low-Throughput (LT) protocol may be used.

Generally, cell free polynucleotides are extracted and isolated by from bodily fluids through a partitioning step in which cell free DNAs, as found in solution, are separated from cells and other non-soluble components of the bodily fluid. Partitioning may include, but is not limited to, techniques such as centrifugation or filtration. In other cases, cells are not partitioned from cell free DNA first, but rather lysed. In this example, the genomic DNA of intact cells is partitioned through selective precipitation. Cell free polynucleotides, including DNA, may remain soluble and may be separated from insoluble genomic DNA and extracted. Generally, after addition of buffers and other wash steps specific to different kits, DNA may be precipitated using isopropanol precipitation. Further clean up steps may be used such as silica based columns to remove contaminants or salts. General steps may be optimized for specific applications. Nonspecific bulk carrier polynucleotides, for example, may be added throughout the reaction to optimize certain aspects of the procedure such as yield.

Isolation and purification of cell free DNA may be accomplished using any means, including, but not limited to, the use of commercial kits and protocols provided by companies such as Sigma Aldrich, Life Technologies, Promega, Affymetrix, IBI or the like. Kits and protocols may also be non-commercially available.

After isolation, in some cases, the cell free polynucleotides are pre-mixed with one or more additional materials, such as one or more reagents (e.g., ligase, protease, polymerase) prior to sequencing.

In some embodiments, the methods of the invention comprise a pre-amplification step to increase the sample number. Thus, prior to the ligation step, the methods comprise annealing a first universal primer to the nucleic acid sequence in the sample, wherein the first universal primer is complementary to a sequence of interest on the nucleic acid sequence and then linearly amplifying the nucleic acid sequence.

In some aspects, the nucleic acid in the sample is fractionated. In some implementations, the methods comprise cleaning up after each amplification step with exonuclease and alkaline phosphatase.

In other aspects, the invention relates to a method of adding oligonucleotide tags to a nucleic acid sequence in a sample, the method comprising the steps of: annealing a first universal primer to the nucleic acid sequence in the sample, wherein the first universal primer is complementary to a sequence of interest on the nucleic acid sequence; linearly amplifying the nucleic acid sequence; and ligating an adapter oligonucleotide to the 3′-end of the nucleic acid sequence, wherein the adapter oligonucleotide comprises: a stem-loop intramolecular nucleotide base pairing; a hydroxyl group at the 3′-end; a phosphate at the 5′-end; a random region complementary to the nucleic acid sequence; and a random region in the loop comprising a molecular barcode

In some embodiments, the linear amplification step comprises annealing a primer to the nucleic acid sequences in the sample and linearly amplifying the nucleic acid sequence. In some implementations, the linear amplification step comprises at least 5 cycles, at least 6 cycles, at least 7 cycles, at least 8 cycles, at least 9 cycles, at least 10 cycles, at least 11 cycles, at least 12 cycles, at least 13 cycles, at least 14 cycles, or at least 15 cycles. In other implementations, the linear amplification step comprises no more than 15 cycles or no more than 10 cycles. For example, the linear amplification step comprises about 10 cycles of amplification.

In some conditions, the intramolecular stem structure of the adapter oligonucleotide has reduced stability where the stem structure is unfolded. In this manner, the stem structure can be designed so that the stem structure can be relieved of its intramolecular base pairing and resemble more of a linear molecule. In one embodiment, the adapter oligonucleotide is designed where the relief of the intramolecular stem structure is thermodynamically favored over the intramolecular stem structure. For example, following the ligation of the adapter oligonucleotide and the nucleic acid sequence, some implementations comprise amplifying the ligated nucleic acid product. The stem-loop structure does not impair the amplification step, because the intramolecular stem structure may be undone by raising the temperature or adding a chemical denaturant. Once the intramolecular stem structure is undone, a probe or primer can be used to sequence or amplify at least a portion of the sequence present in the acceptor molecule. Additional aspects are set forth in International Patent Publication No. WO 2017/205540.

The methods of this disclosure may also enable the cell free polynucleotides to be tagged or tracked in order to permit subsequent identification and origin of the particular polynucleotide. This feature is in contrast with other methods that use pooled or multiplex reactions and that only provide measurements or analyses as an average of multiple samples. Here, the assignment of an identifier to individual or subgroups of polynucleotides may allow for a unique identity to be assigned to individual sequences or fragments of sequences. This may allow acquisition of data from individual samples and is not limited to averages of samples.

In some examples, nucleic acids or other molecules derived from a single strand may share a common tag or identifier and therefore may be later identified as being derived from that strand. Similarly, all of the fragments from a single strand of nucleic acid may be tagged with the same identifier or tag, thereby permitting subsequent identification of fragments from the parent strand. In other cases, gene expression products (e.g., mRNA) may be tagged in order to quantify expression, by which the barcode, or the barcode in combination with sequence to which it is attached can be counted. In still other cases, the systems and methods can be used as a PCR amplification control. In such cases, multiple amplification products from a PCR reaction can be tagged with the same tag or identifier. If the products are later sequenced and demonstrate sequence differences, differences among products with the same identifier can then be attributed to PCR error.

Additionally, individual sequences may be identified based upon characteristics of sequence data for the read themselves. For example, the detection of unique sequence data at the beginning (start) and end (stop) portions of individual sequencing reads may be used, alone or in combination, with the length, or number of base pairs of each sequence read unique sequence to assign unique identities to individual molecules. Fragments from a single strand of nucleic acid, having been assigned a unique identity, may thereby permit subsequent identification of fragments from the parent strand. This can be used in conjunction with bottlenecking the initial starting genetic material to limit diversity.

Further, using unique sequence data at the beginning (start) and end (stop) portions of individual sequencing reads and sequencing read length may be used, alone or combination, with the use of barcodes. In some cases, the barcodes may be unique as described herein. In other cases, the barcodes themselves may not be unique. In this case, the use of non-unique barcodes, in combination with sequence data at the beginning (start) and end (stop) portions of individual sequencing reads and sequencing read length may allow for the assignment of a unique identity to individual sequences. Similarly, fragments from a single strand of nucleic acid having been assigned a unique identity, may thereby permit subsequent identification of fragments from the parent strand.

Generally, the methods and systems provided herein are useful for preparation of cell free polynucleotide sequences to a down-stream application sequencing reaction. Often, a sequencing method is classic Sanger sequencing. Sequencing methods may include, but are not limited to: high-throughput sequencing, pyrosequencing, sequencing-by-synthesis, single-molecule sequencing, nanopore sequencing, semiconductor sequencing, sequencing-by-ligation, sequencing-by-hybridization, RNA-Seq (Illumina), Digital Gene Expression (Helicos), Next generation sequencing, Single Molecule Sequencing by Synthesis (SMSS)(Helicos), massively-parallel sequencing, Clonal Single Molecule Array (Solexa), shotgun sequencing, Maxim-Gilbert sequencing, primer walking, and any other sequencing methods known in the art.

Generally, different sequencing methods provide different coverage of samples. Whole exome sequencing can provide greater depth, but less coverage across the genome, whereas whole genome sequencing can provide a wider coverage across the genome, but at less depth than whole exome sequencing. For example, 200× exome coverage may be the desired coverage for tumor samples, whereas 100× exome coverage may be sufficient for normal samples. Alternatively, 30-40× coverage for whole genome sequencing of tumor samples, with a purity of greater than 0.2, and 20-30× coverage on matched normal samples may be used to provide wider coverage across the genome.

### Cancer Detection and Monitoring

Numerous cancers may be detected and monitored using the methods described herein. Cancers cells, as most cells, can be characterized by a rate of turnover, in which old cells die and replaced by newer cells. Generally dead cells, in contact with vasculature in a given patient, may release DNA or fragments of DNA into the blood stream. This is also true of cancer cells during various stages of the disease. Cancer cells may also be characterized, dependent on the stage of the disease, by various genetic aberrations such as copy number variation as well as rare mutations. This phenomenon may be used to detect the presence or absence of cancers in individuals using the methods described herein.

For example, blood from patients at risk for cancer may be drawn and prepared as described herein to generate a population of cell free polynucleotides. In one example, this might be cell free DNA. The methods of the disclosure may be employed to detect rare mutations or copy number variations that may exist in certain cancers present. The method may help detect the presence of cancerous cells in the body, despite the absence of symptoms or other hallmarks of disease.

The types and number of cancers that may be detected may include but are not limited to blood cancers, brain cancers, lung cancers, skin cancers, nose cancers, throat cancers, liver cancers, bone cancers, lymphomas, pancreatic cancers, skin cancers, bowel cancers, rectal cancers, thyroid cancers, bladder cancers, kidney cancers, mouth cancers, stomach cancers, solid state tumors, heterogeneous tumors, homogenous tumors and the like.

In an embodiment, the cancer is selected from the group consisting of: oral cancer, prostate cancer, rectal cancer, non-small cell lung cancer, lip and oral cavity cancer, liver cancer, lung cancer, anal cancer, kidney cancer, vulvar cancer, breast cancer, oropharyngeal cancer, nasal cavity and paranasal sinus cancer, nasopharyngeal cancer, urethra cancer, small intestine cancer, bile duct cancer, bladder cancer, ovarian cancer, laryngeal cancer, hypopharyngeal cancer, gallbladder cancer, colon cancer, colorectal cancer, head and neck cancer, glioma, parathyroid cancer, penile cancer, vaginal cancer, thyroid cancer, pancreatic cancer, esophageal cancer, Hodgkin's lymphoma, leukemia-related disorders, mycosis fungoides, hematological cancer, hematological disease, hematological malignancy, minimal residual disease, and myelodysplastic syndrome.

In another embodiment, the cancer is selected from the group consisting of: gastrointestinal cancer, prostate cancer, ovarian cancer, breast cancer, head and neck cancer, lung cancer, non-small cell lung cancer, cancer of the nervous system, kidney cancer, retina cancer, skin cancer, liver cancer, pancreatic cancer, genital-urinary cancer, colorectal cancer, renal cancer, and bladder cancer.

In another embodiment, the cancer is non-small cell lung cancer, pancreatic cancer, breast cancer, ovarian cancer, colorectal cancer, or head and neck cancer. In yet another embodiment the cancer is a carcinoma, a tumor, a neoplasm, a lymphoma, a melanoma, a glioma, a sarcoma, or a blastoma.

In one embodiment, the carcinoma is selected from the group consisting of: carcinoma, adenocarcinoma, adenoid cystic carcinoma, adenosquamous carcinoma, adrenocortical carcinoma, well differentiated carcinoma, squamous cell carcinoma, serous carcinoma, small cell carcinoma, invasive squamous cell carcinoma, large cell carcinoma, islet cell carcinoma, oat cell carcinoma, squamous carcinoma, undifferentiated carcinoma, verrucous carcinoma, renal cell carcinoma, papillary serous adenocarcinoma, merkel cell carcinoma, hepatocellular carcinoma, soft tissue carcinomas, bronchial gland carcinomas, capillary carcinoma, bartholin gland carcinoma, basal cell carcinoma, carcinosarcoma, papilloma/carcinoma, clear cell carcinoma, endometrioid adenocarcinoma, mesothelial carcinoma, metastatic carcinoma, mucoepidermoid carcinoma, cholangiocarcinoma, actinic keratoses, cystadenoma, and hepatic adenomatosis.

In another embodiment, the tumor is selected from the group consisting of: astrocytic tumors, malignant mesothelial tumors, ovarian germ cell tumors, supratentorial primitive neuroectodermal tumors, Wilms tumors, pituitary tumors, extragonadal germ cell tumors, gastrinoma, germ cell tumors, gestational trophoblastic tumors, brain tumors, pineal and supratentorial primitive neuroectodermal tumors, pituitary tumors, somatostatin-secreting tumors, endodermal sinus tumors, carcinoids, central cerebral astrocytoma, glucagonoma, hepatic adenoma, insulinoma, medulloepithelioma, plasmacytoma, vipoma, and pheochromocytoma.

In yet another embodiment, the neoplasm is selected from the group consisting of: intraepithelial neoplasia, multiple myeloma/plasma cell neoplasm, plasma cell neoplasm, interepithelial squamous cell neoplasia, endometrial hyperplasia, focal nodular hyperplasia, hemangioendothelioma, and malignant thymoma. In a further embodiment, the lymphoma may be selected from the group consisting of nervous system lymphoma, AIDS-related lymphoma, cutaneous T-cell lymphoma, non-Hodgkin's lymphoma, lymphoma, and Waldenstrom's macroglobulinemia. In another embodiment, the melanoma may be selected from the group consisting of acral lentiginous melanoma, superficial spreading melanoma, uveal melanoma, lentigo maligna melanomas, melanoma, intraocular melanoma, adenocarcinoma nodular melanoma, and hemangioma. In yet another embodiment, the sarcoma may be selected from the group consisting of adenomas, adenosarcoma, chondosarcoma, endometrial stromal sarcoma, Ewing's sarcoma, Kaposi's sarcoma, leiomyosarcoma, rhabdomyosarcoma, sarcoma, uterine sarcoma, osteosarcoma, and pseudosarcoma. In one embodiment, the glioma may be selected from the group consisting of glioma, brain stem glioma, and hypothalamic and visual pathway glioma. In another embodiment, the blastoma may be selected from the group consisting of pulmonary blastoma, pleuropulmonary blastoma, retinoblastoma, neuroblastoma, medulloblastoma, glioblastoma, and hemangiblastomas.

The methods provided herein may be used to monitor already known cancers, or other diseases in a particular patient. This may allow either a patient or practitioner to adapt treatment options in accord with the progress of the disease. In this example, the methods described herein may be used to construct genetic profiles of a particular patient of the course of the disease. In some instances, cancers can progress, becoming more aggressive and genetically unstable. In other examples, cancers may remain benign, inactive, dormant or in remission. The methods of this disclosure may be useful in determining disease progression, remission or recurrence.

Further, the systems and methods described herein may be useful in determining the efficacy of a particular treatment option. In one example, successful treatment options may actually increase the amount of copy number variation or rare mutations detected in patient's blood if the treatment is successful as more cancers may die and shed DNA. In other examples, this may not occur. In another example, certain treatment options may be correlated with genetic profiles of cancers over time. This correlation may be useful in selecting a therapy. Additionally, if a cancer is observed to be in remission after treatment, the systems and methods described herein may be useful in monitoring residual disease or recurrence of disease.

For example, mutations occurring within a range of frequency beginning at threshold level can be determined from DNA in a sample from a patient, e.g., a patient. The mutations can be, e.g., cancer related mutations. The frequency can range from, for example, at least 0.1%, at least 1%, or at least 5% to 100%. The sample can be, e.g., cell free DNA or a tumor sample. A course of treatment can be prescribed based on any or all of mutations occurring within the frequency range including, e.g., their frequencies. A sample can be taken from the patient at any subsequent time. Mutations occurring within the original range of frequency or a different range of frequency can be determined. The course of treatment can be adjusted based on the subsequent measurements.

The present disclosure is further illustrated by the following examples that should not be construed as limiting. The contents of all references, patents, and published patent applications cited throughout this application, as well as the Figures, are incorporated herein by reference in their entirety for all purposes.

### EXAMPLES

### Example 1. Materials and Methods

### DNA Extraction From Tumor and Germline Samples

For one cohort, tumor DNA was extracted from four 10 μm sections obtained from archived formalin-fixed paraffin-embedded tissue using the MAGMAX™ FFPE DNA/RNA ULTRA KIT (ThermoFisher Scientific), following macro-dissection to enrich for tumor cells guided by an H&E stained tumor section. For one cohort, tumor DNA was extracted from ten 30 μm sections obtained from the fresh frozen tumor tissue using the DNeasy Blood and Tissue Kit (Qiagen). Germline DNA was extracted from peripheral blood cells using the DNeasy Blood and Tissue Kit (Qiagen). For one cohort, tumor DNA was extracted from five 10 μm sections obtained from archived formalin-fixed paraffin-embedded tissue using GeneRead DNA FFPE kit (Qiagen). Germline DNA was extracted from peripheral blood cells using the FlexiGene DNA Kit (Qiagen).

### Plasma Processing, DNA Extraction and Quality Assessment

For two cohorts, blood was collected in 10 mL K2 EDTA tubes and centrifuged at 820 g for 10 minutes within 3 hours of venipuncture to separate plasma. 1 mL aliquots of plasma were centrifuged a second time at 16000 g for 10 minutes to pellet any remaining leukocytes and the supernatant plasma was stored at −80 ° C. For one cohort, blood was collected in Streck cell-free BCT tubes (Streck) and centrifuged twice to separate plasma. The first spin was at 1600 g for 15 minutes at 25° C. The plasma was then aliquoted and centrifuged again for 10 minutes at 2500 g at 25° C. cfDNA was extracted using either the QIAsymphony DSP Circulating DNA Kit (Qiagen) or MagMAX Cell-Free DNA Isolation kit (ThermoFisher Scientific). All cfDNA samples were evaluated for yield and quality using droplet digital PCR, as described previously (27).

### Tumor/Germline Exome Sequencing

For two cohorts, tumor/germline exome sequencing libraries were prepared using the KAPA Hyper Prep Kit following manufacturer's instructions. Exome enrichment through hybridization was performed using a customized version of Agilent SureSelect V6 exome. For one cohort, tumor and germline exome libraries were generated using the Illumina NEXTERA™ RAPID CAPTURE EXOME LIBRARY PREPARATION KIT. We pooled exome libraries and sequenced on ILLUMINA® HISEQ.

### Variant Calling in Tumor Exomes and Identification of Target Mutations

Reads were aligned to human genome version hg19 using bwamem (28), followed by base recalibration using GATK (29), duplicate identification using Picard tools MarkDuplicates, and indel realignment using GATK. Germline mutations were inferred using GATK HaplotypeCaller and Freebayes (30). Somatic tumor mutations were called using MuTect (31), Seurat (32) and Strelka (33). Somatic mutations with an allele frequency <5% were removed.

### Identification of Putative Founder Mutations

Potential target mutations found on autosomal chromosomes were assessed for copy number, purity, and variant allele frequency (VAF). We used Sequenza to infer both the proportion of tumor cells in the sequenced tumor DNA sample and copy number alterations in the tumor (34). For each mutation, the mean variant allele frequency from the variant callers, sample purity, and local copy number were used to infer its cancer cell fraction (CCF) via two different methods: an implementation of the algorithm from McGranahan et al. (35), and PyClone (36). For each sample, the VAF, minor and major copy number, and purity were used as input for PyClone analysis with 25,000 iterations, including 10,000 iterations of burn in.

Founder mutations were identified using a set of criteria for mutation confidence and maximum CCF. To quality as a target for ctDNA analysis, a mutation must have been identified by at least 2 somatic mutation callers, have a mean of >20× germline reads passing each mutation caller's filters that covered the mutated base, a germline VAF <0.01%, and >50× mean tumor passing filter reads. In addition, the upper range of the CCF distribution calculated using the McGranahan et al. approach must overlap with 1.0, and the mutation must be found in the highest CCF PyClone mutation cluster.

### Primer Design for TARDIS

Mutations that passed the filtering steps above were used as targets for TARDIS primer design. The primer design process is focused on maximizing TARDIS performance and minimizing spurious amplification, particularly in the linear pre-amplification stage. We first generated primers on the forward or reverse strands up to 350 bp from the target mutation position for both linear and exponential amplification reactions (Primers 1 and 2 for each targeted locus) (37). Primer 1 melting temperature (Tm) range was set to 68-74° C., and Primer 2 Tm range was 56-60° C., with Primer 1 upstream and a maximum of 3 bp overlap allowed between Primers 1 and 2. During primer selection, we minimized the distance between the 3′ end of Primer 2 and the target mutation position, to ensure short mutant molecules are captured efficiently. To avoid erroneous variants caused by primer synthesis overhangs, we also required a minimum 3 bp distance to the target mutation. To avoid unintended amplification in multiplexed PCR reactions, we used a combination of in silico PCR, sequence comparison to the genome using LAST (38), and 3′ primer kmer matching to identify problematic primers for multiplexing. Primer is with more than 2 LAST matches outside the target region are excluded, along with Primer 2s with any LAST off target matches. All combinations of potential Primer is are analyzed using in silico PCR. Next, a graph is built in which nodes represent primers and edges link pairs of primers with predicted PCR products. The nodes are sorted by number of edges, and we iteratively remove the node with the most edges if it is not the last Primer 1 for a given target. This process continues until there are no remaining edges or until all targets only have a single Primer 1 remaining. If there are multiple remaining Primer 1s for a given target, the one with the fewest kmer matches to other target regions is selected. This process is repeated for Primer 2s, except the best primer after graph analysis is selected based on minimizing distance to the target mutation rather than kmer matches. A test run of TARDIS using each new primer panel was conducted with 8 replicates of sheared genomic DNA before analyzing plasma samples to identify any remaining problematic primers. The proportion of soft masked reads, the proportion of total reads in the library generated from products of that primer, and the proportion of reads in the most abundant molecule size were calculated for each primer. A target was removed from the panel prior to analysis of plasma samples if the median proportion of soft masked reads across replicates is >0.5, if the maximum proportion of soft masked reads in any replicate is >0.75, if the median read proportion across replicates for the primer is >4*(1/total primer count), if the max proportion in any replicate is >0.75, if the median proportion of reads in the most abundant size is >0.25, if the max proportion in the most abundant size in any replicate is >0.5, or if the median number of molecule sizes is <20.

### Preparation of TARDIS Sequencing Libraries

TARDIS sequencing libraries were prepared using target-specific linear pre-amplification, ligation, 1-2 rounds of target-specific exponential amplification and barcoding PCR. TARDIS reactions were set up using up to 20 ng of template plasma DNA in 10 μL volume for linear pre-amplification. For each TARDIS run, patient-specific primers were pooled equimolarly. For pre-amplification, each Primer 1 pool was used at a final concentration of 0.5-1.0 μM (regardless of the number of primers in the panel). Linear pre-amplification was performed using Kapa HiFi HotStart ReadyMix (Kapa Biosystems) at the following thermocycling conditions: 95° C. for 5 minutes followed by 50 cycles at 98° C. for 20 seconds, 70° C. for 15 seconds, 72° C. for 15 seconds, and 72° C. for 1 minute. This reaction was followed by a magnetic bead cleanup (SPRIselect, Beckman Coulter) at 1.8× ratio after addition of 10% ethanol. Pre-amplified DNA was eluted in 10 μL water. After dephosphorylation using FastAP (ThermoFisher Scientific), 0.8 μL of 100 μM ligation adapter was added to each sample. The sequence of the hairpin oligonucleotide used for single-stranded DNA ligation is provided in Table 1 and was adapted from Kwok et al. (39). Samples were denatured at 95° C. for 5 minutes and immediately transferred to an ice bath for at least 2 minutes. We setup ligation reactions using 2.5 μL 10× T4 DNA Ligase buffer (New England Biolabs), 2.5 μL of 5 M betaine, 2,000 U of T4 DNA ligase (New England Biolabs) and 5.8 μL of 40%-60% PEG8000. Ligation was performed at 16° C. for 16-24 hours. A magnetic bead cleanup (SPRIselect) was performed at 1× buffer ratio after initially diluting the sample by adding 20-40 μL water (to reduce effective PEG concentration during cleanup). An additional dephosphorylation was performed using FastAP.

TABLE 1
Oligonucleotide (primer) sequences used during TARDIS library preparation.

| Step | Oligonucleotide sequence |
|---|---|
| Primer 1 | Target-specific primers with no specific modifications |
| (used for Linear Pre-Amplification and | |
| Round 1 Exponential Amplification) | |
| Ligation | Hairpin oligonucleotide: |
| | 5′-Phos-TGCTCTTTG-9 bp UMI-GTCAGATCGGAAGAGCA-NNNNNN- |
| | CTGCCCATAGAG-SpC3-3′ (SEQ ID NO: 1) |
| Primer 2 | Target-specific Forward Primers with 5′ tag: |
| (used for Round 2 Exponential | 5′-CGCTCTTCCGATCTCTG-target-specific primer-3′ (SEQ ID NO: 2) |
| Amplification) | Universal Reverse Primer: |
| | 5′-GTGACTGGAGTTCAGACGTGTGCTCTTCCGATCTGAC-3′ (SEQ ID NO: 3) |
| Barcoding PCR (Addition of sample | Sample Barcode Forward Primers: |
| indexes) | 5′-CAAGCAGAAGACGGCATACGAGAT-6 bp sample index- |
| | GTGACTGGAGTTCAGACGTGTGCTCTTCCGATCTGAC-3′ (SEQ ID NO: 4) |
| | Universal Reverse Primer |
| | 5′-AATGATACGGCGACCACCGAGATCTACACTCTTTCCCTAC |
| | ACGACGCTCTTCCGATCTCTG-3′ (SEQ ID NO: 5) |
| Illumina | Custom Sequencing Primer 1: |
| Sequencing | 5′-ACACTCTTTCCCTACACGACGCTCTTCCGATCTCTG-3′ (SEQ ID NO: 6) |
| | Custom Sequencing Primer 2: |
| | 5′-GTGACTGGAGTTCAGACGTGTGCTCTTCCGATCTGAC-3′ (SEQ ID NO: 7) |

Exponential PCR was performed in two rounds. In both rounds, a universal reverse primer was used, complementary to the ligated adapter and upstream of the UMI (see Table 1 for primer sequences). On the target-specific end, Primer 1 pools were used for the first round and Primer 2 pools were used for the second round. When total number of targeted mutations exceed 30, 2 μL of amplified DNA from round 1 was split across multiple round 2 reactions of ˜30 targets each. In a subset of samples, only the second round of exponential amplification was performed using total ligated DNA. Primers were pooled equimolarly and used at a final pool concentration of 0.5 μM. Round 1 amplification was performed using Kapa HiFi HotStart ReadyMix with the following thermocycling conditions: 95° C. for 5 minute followed by 5 cycles at 98° C. for 20 seconds, 65° C. for 2 minutes, and 15 cycles at 98° C. for 20 seconds, 65° C. for 15 seconds and 72° C. for 15 seconds, followed by a 1 minute incubation at 72° C. Round 2 amplification was performed using NEBNext Q5 Hot Start HiFi PCR Mastermix (New England Biolabs) with the following thermocycling conditions: 98° C. for 1 minute followed by 5 cycles at 98° C. for 10 seconds, 61.5° C. for 4 minutes, and 15 cycles at 98° C. for 10 seconds, 61.5° C. for 30 seconds and 72° C. for 20 seconds, followed by a 2 minute incubation at 72° C. Intervening and final magnetic bead cleanups were performed at 1.7× volume ratio (SPRIselect) and product were eluted in 20-40 μL water.

Barcoding PCR was performed using universal primers to introduce sample specific barcodes and complete sequencing adapters, as described previously (14). We used 1 U per reaction of Platinum Taq DNA Polymerase High Fidelity (Invitrogen) in the following buffer: 1.3× Platinum buffer, 0.4M betaine, 2.5 μL/r×n of DMSO, 0.45 mM dNTPs, 1.75 mM MgSO4 and primers at 0.5 μM. 10 μL of the product from exponential amplification was used as template, at the following thermocycling conditions: 94° C. for 2 minutes followed by 15 cycles at 94° C. for 30 seconds, 56° for 30 seconds, 68° C. for 1 minute, and a final incubation at 68° C. for 10 minutes. A final magnetic bead cleanup (SPRIselect) was performed at 1.2× volume ratio. TARDIS libraries were eluted in 20 μL DNA suspension buffer, quantified using fluorometric and electrophoretic assays and pooled for sequencing. Sequencing was performed on Illumina HiSeq 4000 or Illumina NextSeq.

### Analysis of TARDIS Sequencing Data

TARDIS amplicon sequencing reads were aligned to human genome hg19 using bwa-mem. Read pairs whose R1 read mapped to the start position of a target primer were considered on-target reads, while the position of the R2 read was used to determine the length of the template molecule. The UMI sequence and molecule size were used to identify all of the reads that came from the same template molecule. To minimize incorrect assignment of reads to read families, we implemented a directed adjacency graph approach inspired by Smith et al. (40). Briefly, a graph is constructed in which each UMI is a node and an edge was designated from node A to node B where the two nodes UMI sequences differ by one base, and node A's read count is at least 2× node B. All of the reads from UMIs in each component from the resulting graph constitute a read family and are considered to have come from the same original molecule. UMI variation within a read family is assumed to arise due to PCR or sequencing error. We found that a small number of UMIs with very few reads had incoming edges from multiple otherwise separate components. The component assignment of these nodes is ambiguous, and they significantly reduced the number of independent components in the graph. To resolve this issue, any UMI that had two or more incoming edges and no outgoing edges was removed. We then inferred the allele at the target position by consensus of all R1 reads in a given component, requiring that at least 90% of the R1 reads carried a particular allele at the position of interest. In practice, the vast majority of read families contained fewer than 10 reads, and therefore required perfect agreement at the target position. Inferred molecules with less than 90% read support for a variant were removed as inconclusive.

To ascertain ctDNA detection in a sample, we required support of at least 2 RFs across all mutations covered by at least 100 Total RFs. For any mutations supporting ctDNA detection, we required that its AF (Mutant RFs/Total RFs) represent at least 0.5 mutant molecules in the reaction. In addition, the ratio between number of RFs supporting a mutation and mixed RFs observed at that locus must be <10. If only one mutation supported ctDNA detection, we required at least 2 independent RF sizes (to ensure at least two unique ligated molecules). This requirement was waived if >1 mutation supported ctDNA detection. For each mutation observed, the probability of encountering the number and fragment sizes of mutant RFs was calculated using a distribution of background errors (see below). For each sample, the combined probability of mutations detected was calculated and corrected for multiple testing using the Bonferroni approach to account for number of mutations analyzed in each TARDIS panel. Any other multiple-testing correction approach may also be applied. Sample-level ctDNA detection was confirmed if Bonferroni corrected p-value was <0.05. The p-value threshold may be adjusted as required to be <0.01, <0.005, <0.001 as required. Since not all sequenced molecules may receive enough reads to form read families, allele fraction (AF) for a given mutation was calculated as the proportion of all reads that contained the target variant. To quantify ctDNA levels in a sample, we calculated mean AFs over all targeted mutations. However, to avoid the contribution of background noise, AFs for any mutations not supported by ≥1 mutant RFs, a ratio of mutant RFs with Mixed RFs of ≥10 or <0.5 mutant molecules were set to zero prior to calculating the mean.

### TARDIS Analysis Pipelines

Target selection and primer design pipelines were developed in Python3 using NumPy, SciPy, networkX, pandas, and matplotlib, and in Julia 0.6.2 using BioJulia, DataFrames, Gadfly, and LightGraphs. Data analysis and plotting were conducted in Python3, Julia 1.1, and R v3 using ggplot2.

### Calculation of Background Error Rates

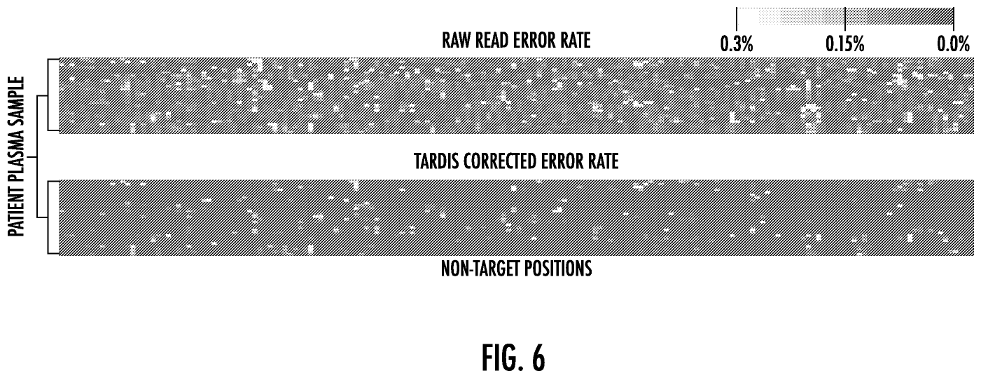

To measure overall background error rates, we evaluated the first 10 bp from a set of amplicons across multiple representative plasma samples for highest non-reference alleles (starting 3 bp downstream of target-specific primers), excluding the targeted locus. The full dataset included 200 loci from each of 39 samples, for a total of 7,800 independent positions. In raw sequencing results, we observed a mean error rate per base of 6.4×10−4 and median error rate per base of 2.2×10−4, with background errors observed at 77% of tested positions. Similar to detection of individual variants described above, we required consensus of all members of an RF, a minimum of 2 RFs with a ratio between variant RFs and mixed RFs <10 and found a significantly reduced mean error rate of 1.1×10−4 and a median of 0 (FIG. 6). 93% of tested positions were error-free. Unlike variant calling for genotyping, TARDIS relies on detection and quantification of pre-identified non-reference alleles at pre-selected genomic loci. We do not call mutations at every sequenced locus, lowering the probability of false positives. Moreover, given limited template DNA input, individual mutations are not expected at AFs below or close to the background error rate using RF consensus. However, analysis of dozens or hundreds of mutations could result in false positive ctDNA detection due to multiple testing. To ensure confidence in sample-level ctDNA detection, we used a background distribution of number of non-reference RFs and number of fragment sizes for randomly chosen non-reference alleles at non-target loci (3-30 bp from end of sequenced primer) to calculate the probability of observed each mutation in a sample. We calculated a combined probability for each sample as the product of probabilities for observed mutations and applied multiple testing correction using the Bonferroni approach, requiring the correct p-value to be <0.05.

### Example 2. Development of TARDIS and Evaluation of Assay Performance in Reference Samples

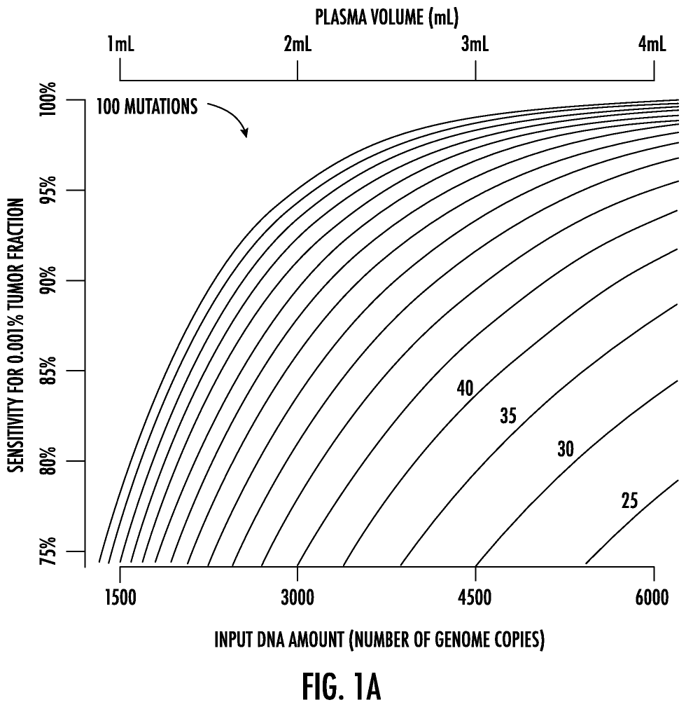

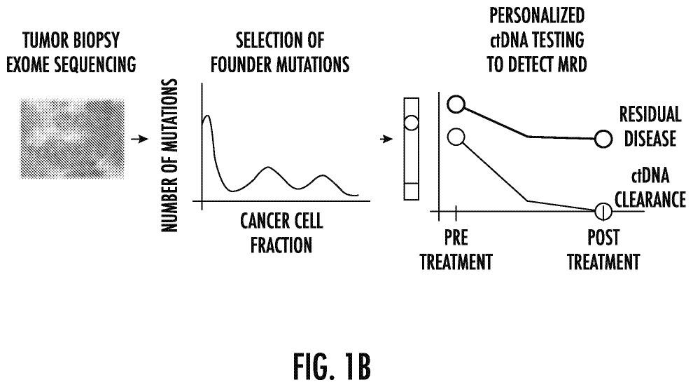

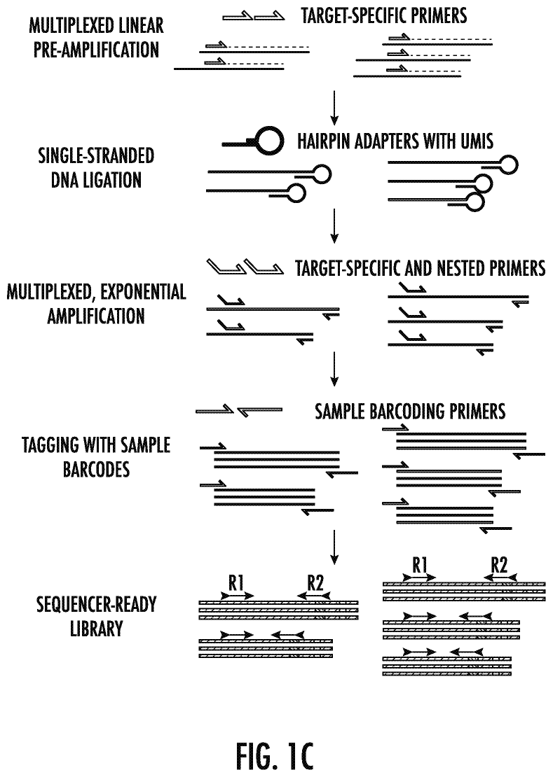

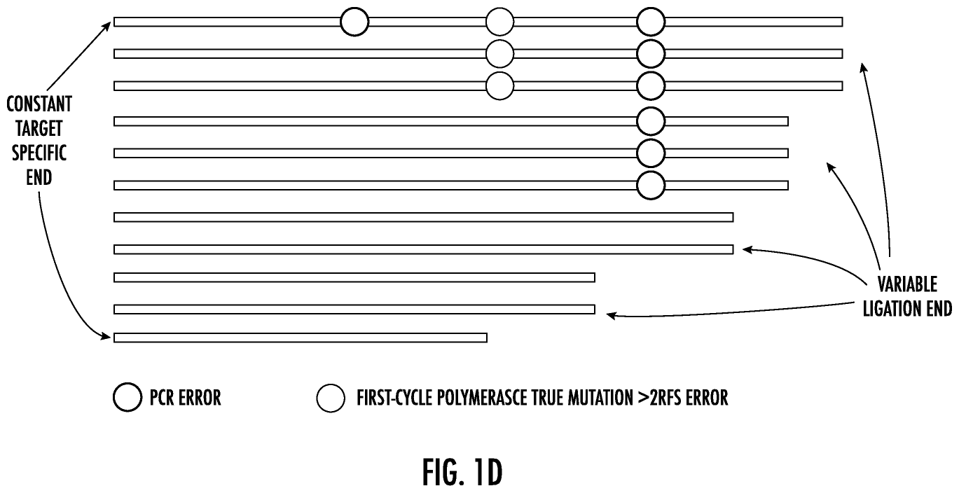

We developed TARDIS to improve analytical sensitivity and quantitative precision for ctDNA analysis by maximizing interrogation of tumor-derived DNA fragments in limited amounts of plasma DNA. To achieve this, we leverage simultaneous deep sequencing of patient-specific somatic mutations while minimizing template DNA losses during library preparation and suppressing background errors. For each patient, we identify putative founder somatic mutations using exome sequencing of tumor biopsies and analyze dozens to hundreds of mutations simultaneously in serial plasma DNA samples obtained during treatment (FIG. 1B). To maximize capture and analysis of input DNA while preserving specificity, we perform targeted linear pre-amplification, followed by single-stranded DNA ligation with unique molecular identifiers (UMIs), targeted exponential PCR and sequencing (FIG. 1C). The resulting sequencing reads at each targeted locus have a fixed amplification end and a variable ligation end, preserving fragment size information unlike conventional PCR amplicons (13, 14). We utilize fragment sizes and UMIs to group sequencing reads into read families (RFs) and require consensus of all members to distinguish true low abundance mutations from polymerase or sequencing background errors (FIG. 1D).

To evaluate analytical performance of TARDIS at low ctDNA levels, we designed a multiplexed panel targeting 8 mutations in commercially available reference samples for cfDNA analysis (Table 2). We analyzed a total of 93 replicates, 7-16 each at 1%, 0.5%, 0.25%, 0.125%, 0.063%, 0.031% Allele Fractions (AFs) and 16 wild-type (WT) samples. AFs for individual mutations were verified by droplet digital PCR (ddPCR) by the vendor (except for 0.063% and 0.031% that were dilutions of 0.125% in WT, Table 3). Input DNA in each replicate was 5.6-7.9 ng (1682-2394 haploid genomic equivalents). Mean number of mutated molecules expected for each targeted mutation in a sample was 0.90-19.6 across 0.031%-1% AFs.

TABLE 2
Mutations targeted in reference samples. 8 mutations include 6 SNVs,
one 15 bp deletion and one insertion.

| Gene | Mutatio | Chr | Position | Ref | Alt | Primer 1 | Primer 2 | Prime |
|---|---|---|---|---|---|---|---|---|
| AKTI | SNV | 14 | 105246551 | C | T | AGGTGCCATCATTCTTGAGGAG | CCGCCAGGTCTTGATGT | + |
| | | | | | | GAA (SEQ ID NO: 8) | (SEQ ID NO: 9) | |
| EGFR_1 | 15 bp | 7 | 55242466 | G | — | TGTCATAGGGACTCTGGATCCCA | AGTTAAAATTCCCGTCGCT | + |
| | Deletio | | | | | GAA (SEQ ID NO: 10) | AT (SEQ ID NO: 11) | |
| EGFR_2 | SNV | 7 | 55249071 | C | T | CGTGGACAACCCCCACGTGT (SEQ | CCACCGTGCAGCTCA (SEQ | + |
| | | | | | | ID NO: 12) | ID NO: 13) | |
| GNA11 | SNV | 19 | 3118942 | A | T | TGGCTGAGTCCTGGCGCTGT (SEQ | TTTCAGGATGGTGGATGTG | + |
| | | | | | | ID NO: 14) | (SEQ ID NO: 15) | |
| IDH1 | SNV | 2 | 209113113 | G | A | TGCAAAAATATCCCCCGGCTTG | GGATGGGTAAAACCTATCA | − |
| | | | | | | (SEQ ID NO: 16) | TCA(SEQ ID NO: 17) | |
| PDGFRA | SNV | 4 | 55152093 | A | T | GGGAAGTGAGGACGTACACTGCC | ACATAGTTCGAATCATGCA | − |
| | | | | | | TTT (SEQ ID NO: 18) | TG (SEQ ID NO: 19) | |
| PIK3CA | 1 bp | 3 | 178952149 | C | C[insA] | GCTGGACAACAAAAATGGATTGG | CTTCCACACAATTAAACAG | + |
| | Insertion | | | | | ATCTT (SEQ ID NO: 20) | CA(SEQ ID NO: 21) | |
| RET | SNV | 10 | 43617416 | T | C | ACACTTACACATCACTTTGCGTGG | TGATCAAAAAGGGATTCAA | − |
| | | | | | | TGTAGA (SEQ ID NO: 22) | TT (SEQ ID NO: 23) | |

TABLE 3
Expected mutation levels in reference samples analyzed. Reference
samples were obtained at 1%, 0.5%, 0.25%, 0.125% and WT (Seraseq
ctDNA Mutation Mix v2, Seracare). Reference samples at 0.063%
and 0.031% were prepared as dilutions of 0.125% in the WT sample.
Known AFs measured by droplet digital PCR (ddPCR) as reported
by vendor for 0.125%-1% AF levels and calculated using 0.125%
AFs for 0.031% and 0.063% AF levels, using dilution factors.

| | | | | Known | Number of |
|---|---|---|---|---|---|
| | Input per | Input per | | Mutation | Mutant |
| | reaction | reaction | | AF | Molecules |
| AF level | (hGEs) | (ng) | Primer | (ddPCR) | Expected |
| 1.000% | 1727 | 5.7 | AKT1 | 1.050% | 18.1 |
| | | | EGFR_1 | 1.160% | 20.0 |
| | | | EGFR_2 | 1.130% | 19.5 |
| | | | GNA11 | 1.380% | 23.8 |
| | | | IDH1 | 1.330% | 23.0 |
| | | | PDGFRA | 1.130% | 19.5 |
| | | | PIK3CA | 0.850% | 14.7 |
| | | | RET | 1.070% | 18.5 |
| 0.500% | 2030 | 6.7 | AKT1 | 0.530% | 10.8 |
| | | | EGFR_1 | 0.560% | 11.4 |
| | | | EGFR_2 | 0.540% | 11.0 |
| | | | GNA11 | 0.690% | 14.0 |
| | | | IDH1 | 0.750% | 15.2 |
| | | | PDGFRA | 0.530% | 10.8 |
| | | | PIK3CA | 0.470% | 9.5 |
| | | | RET | 0.550% | 11.2 |
| 0.250% | 1682 | 5.5 | AKT1 | 0.280% | 4.7 |
| | | | EGFR_1 | 0.300% | 5.0 |
| | | | EGFR_2 | 0.320% | 5.4 |
| | | | GNA11 | 0.340% | 5.7 |
| | | | IDH1 | 0.360% | 6.1 |
| | | | PDGFRA | 0.260% | 4.4 |
| | | | PIK3CA | 0.230% | 3.9 |
| | | | RET | 0.280% | 4.7 |
| 0.125% | 2394 | 7.9 | AKT1 | 0.160% | 3.8 |
| | | | EGFR_1 | 0.120% | 2.9 |
| | | | EGFR_2 | 0.180% | 4.3 |
| | | | GNA11 | 0.150% | 3.6 |
| | | | IDH1 | 0.190% | 4.5 |
| | | | PDGFRA | 0.140% | 3.4 |
| | | | PIK3CA | 0.130% | 3.1 |
| | | | RET | 0.140% | 3.4 |
| 0.063% | 2379 | 7.9 | AKT1 | 0.080% | 1.9 |
| (dilution of | | | EGFR_1 | 0.060% | 1.4 |
| 0.125% in | | | EGFR_2 | 0.090% | 2.1 |
| WT) | | | GNA11 | 0.075% | 1.8 |
| | | | IDH1 | 0.095% | 2.3 |
| | | | PDGFRA | 0.070% | 1.7 |
| | | | PIK3CA | 0.065% | 1.5 |
| | | | RET | 0.070% | 1.7 |
| 0.031% | 2371 | 7.8 | AKT1 | 0.040% | 0.9 |
| (dilution of | | | EGFR_1 | 0.030% | 0.7 |
| 0.125% in | | | EGFR_2 | 0.045% | 1.1 |
| WT) | | | GNA11 | 0.038% | 0.9 |
| | | | IDH1 | 0.048% | 1.1 |
| | | | PDGFRA | 0.035% | 0.8 |
| | | | PIK3CA | 0.033% | 0.8 |
| | | | RET | 0.035% | 0.8 |

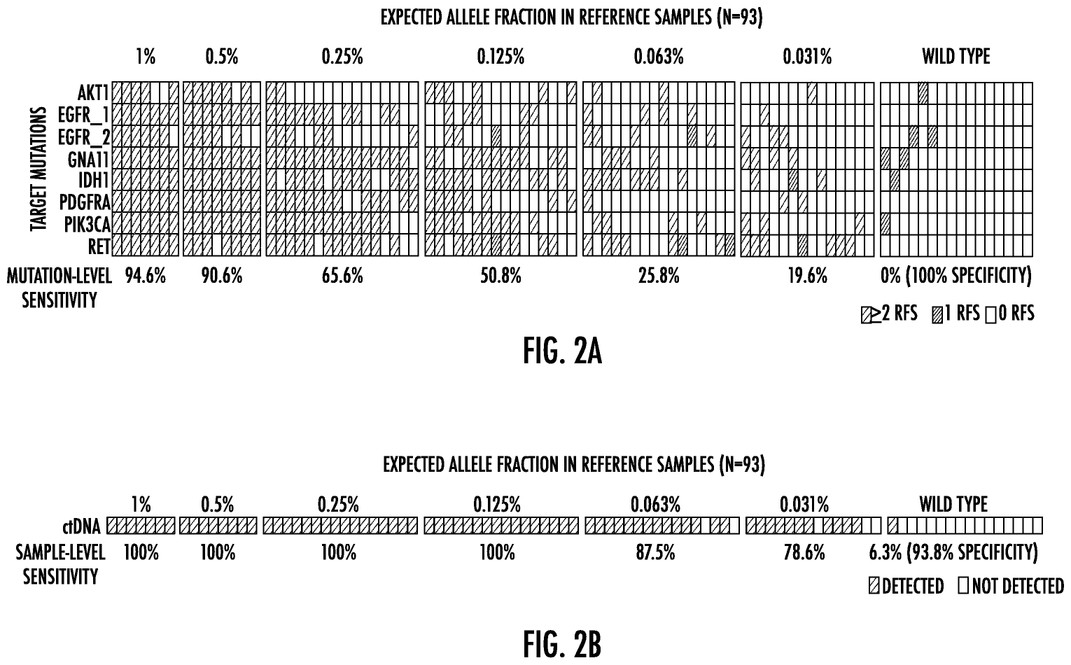

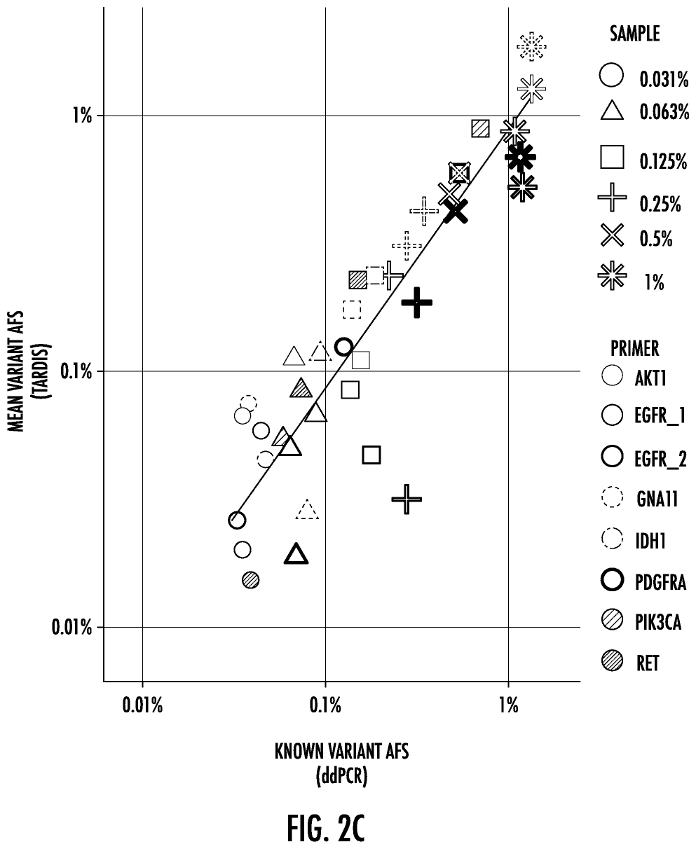

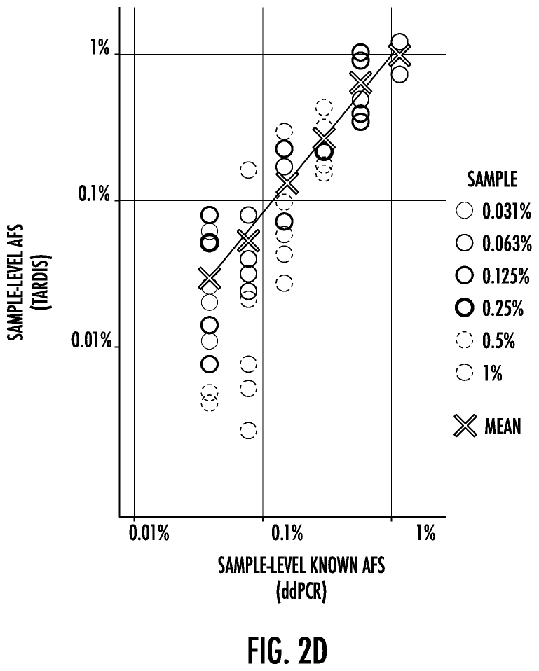

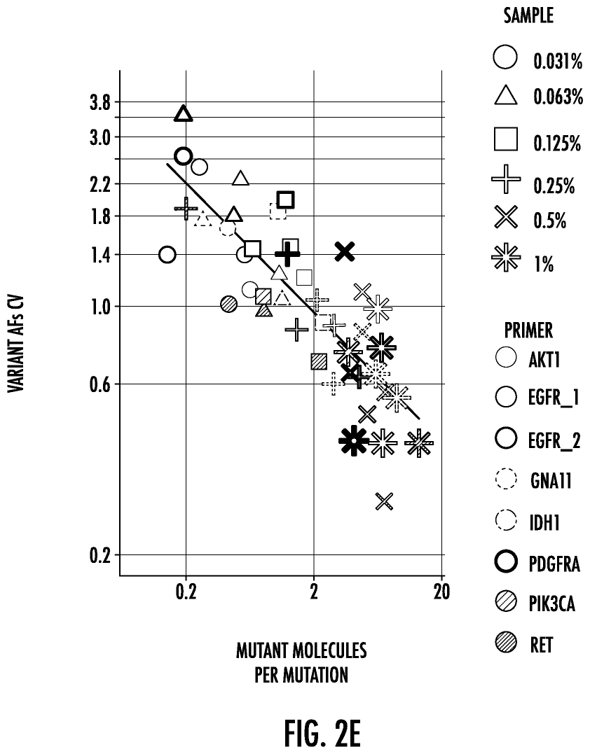

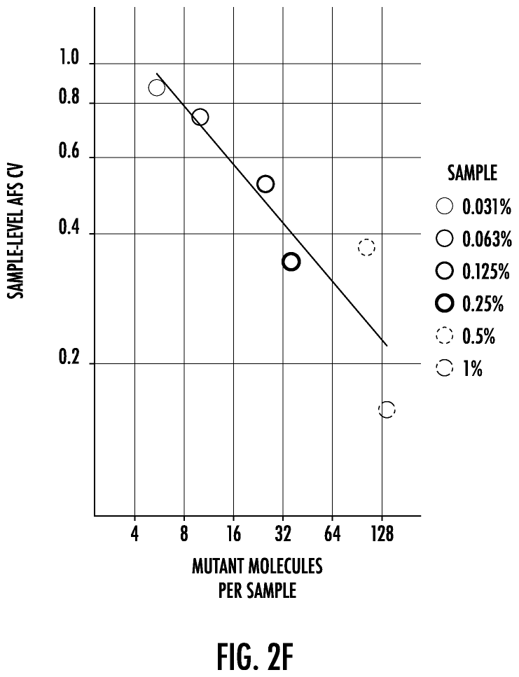

To exclude polymerase errors introduced during linear or exponential amplification, we required at least two independent DNA fragments (≥2 RFs) and measured AF consistent with ≥0.5 mutant molecules to support each variant call. In reference samples, we achieved mutation-level sensitivity of 94.6%, 90.6%, 65.6%, 50.8%, 25.8% and 19.6% respectively at 1%, 0.5%, 0.25%, 0.125%, 0.063% and 0.031% AFs, consistent with decreasing number of mutant molecules at lower AFs (FIG. 2A, Data S1). Using the same criteria, none of the 128 candidate mutations were detected in wild-type samples (100% specificity). Analogous to the detection of tumor-derived DNA in plasma, we leveraged multiple mutations to evaluate sample-level sensitivity. To ascertain mutant DNA in a sample, we required ≥2 RFs contributed by one or more mutations, each with measured AF consistent with ≥0.5 mutant molecules in input DNA. In samples where a single mutation was detected, we required supporting RFs with ≥2 fragment sizes. We achieved sample-level sensitivity of 100% for 0.125%4% AFs, 87.5% for 0.063% and 78.6% for 0.031% AF (FIG. 2B). Using the same criteria, we detected mutant DNA in 1 of 16 wild-type samples (93.8% specificity). These results demonstrate the principle underlying TARDIS i.e. leveraging multiple patient-specific mutations to overcome limits of sampling and to improve limit of detection. We successfully detected mutant DNA in 11 of 14 replicates with 0.031% AF with 7.8 ng input DNA per reaction, when we expected a total of 7.2 mutant molecules per reaction across 8 mutations (<1 mutant molecule per mutation).

To determine quantitative accuracy, we compared known AFs for variants measured by ddPCR in reference samples to mean AFs measured using TARDIS and found a strong correlation (Pearson r=0.921, p<2.2×10−16, FIG. 2C). To evaluate agreement between observed and expected mutant fraction in each sample (equivalent to ctDNA fraction in plasma samples), we calculated sample-level mean AFs (mean of 8 mutations in each replicate) and found an excellent correlation between observed and expected AFs (Pearson r=0.937, p<2.2×10−16, FIG. 2D). To evaluate quantitative precision, we calculated coefficient of variation (CV) for mutation AFs across replicates and expected AF levels (FIG. 2E). Variant-level CVs were strongly correlated with expected number of mutated molecules (Pearson r=−0.854, p=1.2×10−14), ranging from 0.28 (17.8 average mutant molecules per mutation) to 3.74 (0.08 mutant molecules per mutation). A similar trend was observed in CVs for sample-level mean AFs (Pearson r=−0.936, p=0.006), although precision improved when multiple mutations were aggregated together. Sample-level CVs ranged from 0.16 for 1% expected AF (137.9 average mutant molecules per reaction) to 0.87 for 0.031% expected AF (5.4 mutant molecules per reaction, FIG. 2F).

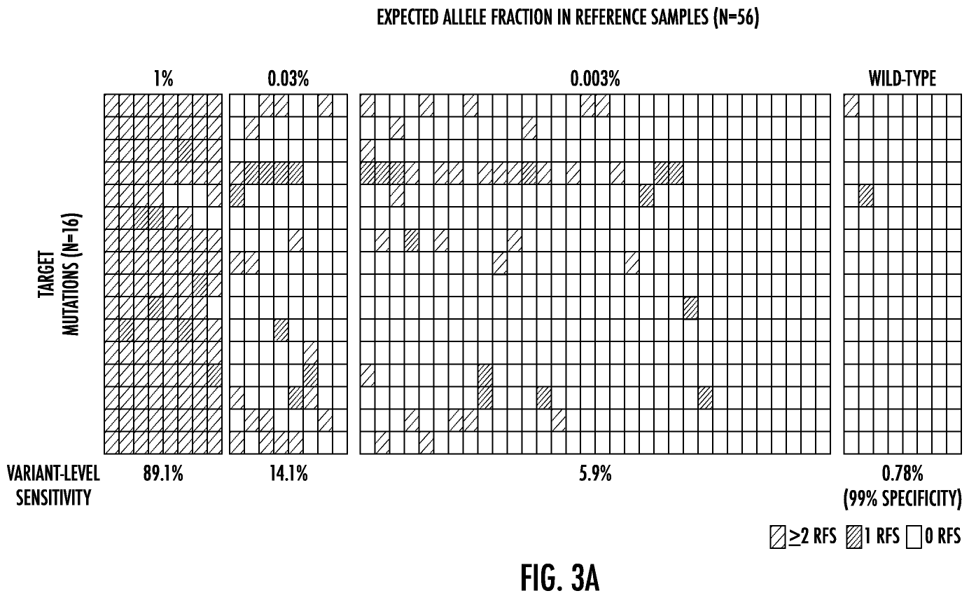

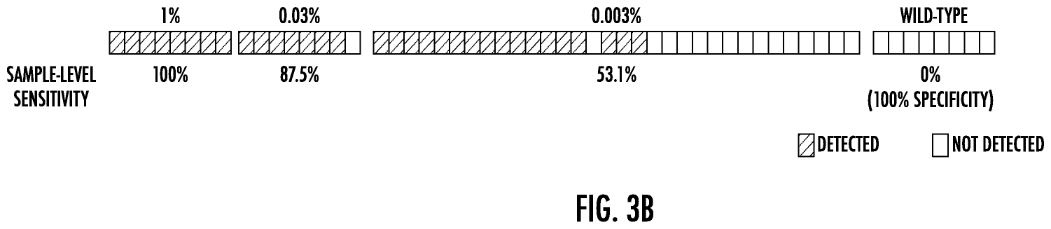

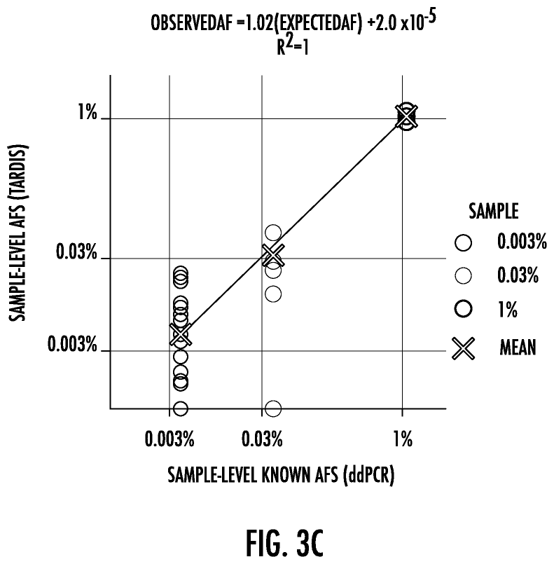

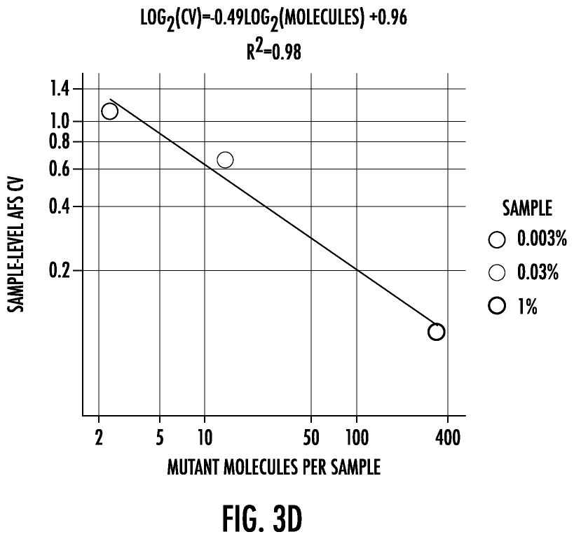

To evaluate if we can improve the limit of detection further using clinically accessible amounts of plasma DNA, we performed an additional experiment targeting 16 mutations in 56 replicates from reference samples, 8 replicates each at 1%, 0.03% AFs and wild- type and 32 replicates at 0.003% AF. DNA input per reaction was 5.0-13.6 ng for 1%, 0.03% and wild-type samples while 20.0-27.2 ng for 0.003% AF. Using these input amounts, we expected an average of 38.1, 1.6 and 0.28 mutant molecules per mutation and detected 89.1%, 14.1% and 5.9% in 1%, 0.03% and 0.003% AFs respectively (FIG. 3A, Data S2). Only 1 of 128 candidate mutations were erroneously called as false positive in wild-type samples (99.2% specificity). Aggregating 16 mutations together, we expected 553, 19.8 and 2.9-4.0 total mutant molecules per sample. At the sample-level, we detected tumor DNA in 8/8 (100%), ⅞ (87.5%) and 17/32 (53.1%) replicates in 1%, 0.03% and 0.003% AFs respectively (FIG. 3B). Tumor DNA was not confidently detected in any of 8 wild-type samples using our established criteria (see Methods in Example 1). To mimic analysis of cfDNA equivalent to two 10 mL blood tubes (still clinically accessible and relevant volume), we tested all possible combinations of any 2 replicates at 0.003% to achieve a sample-level sensitivity of 85.9% (237/276 combinations). Using a similar approach with wild-type samples, we detected tumor DNA in 1/28 combinations (96.4% specificity). As in the previous 8 mutations experiment, we observed excellent agreement between sample-level AFs measured using TARDIS and digital PCR (FIG. 3C). Overall, these results confirm quantitative accuracy of TARDIS and demonstrate that simultaneously assaying multiple patient-specific mutations improves sensitivity as well as quantitative precision.

Since limited blood volumes can be obtained clinically, a key performance metric for ctDNA assays is conversion efficiency i.e. the fraction of input DNA molecules that are successfully analyzed. TARDIS uses several cycles of linear pre-amplification prior to ligation with UMIs and therefore, we expect the number of read families to be several folds higher than input. To estimate effective molecular conversion for TARDIS, we leveraged multiple replicates from reference samples and inferred effective conversion by comparing observed performance (sensitivity and precision) and expected performance (based on the Poisson distribution), given expected mutation AFs, input levels and sequencing coverage. Measuring 16 candidate mutations in aggregate, we found precision improved as the number of total mutant molecules increased in the reaction (FIG. 3D). Theoretically, a linear fit to log2(CV) as a function of log2(expected number of mutant molecules) should have a slope of −0.5, with CVs decreasing as the inverse square root of the number of rare mutant molecules (1/√n) increases. In our data, we observed a slope of −0.496, consistent with this expectation. Using the intercept for the linear fit and input amounts used for each replicate, we inferred an effective molecular conversion rate of 26%. In contrast, the sample-level sensitivity observed across 32 replicates of 0.003% suggests effective molecular conversion rate of ˜39%, based on expected AFs. While previous studies have not evaluated effective conversion similarly using real world performance metrics, our estimates surpass current experience with most sequencing-based plasma DNA assays that utilize UMIs or other error suppression strategies. The discrepancy between the two conversion estimates for our data likely results from higher estimates of CVs due to variable conversion efficiencies between targeted mutations. These could result from differences in efficiency of linear and exponential amplification, fragment sizes, local secondary structures and depth of sequencing coverage.

### Example 3. Detection of Residual Disease in Patients with Early and Locally Advanced Breast Cancer

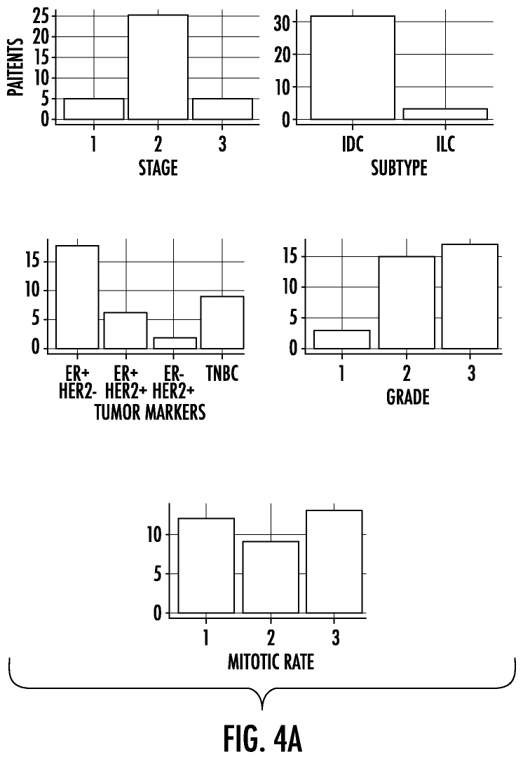

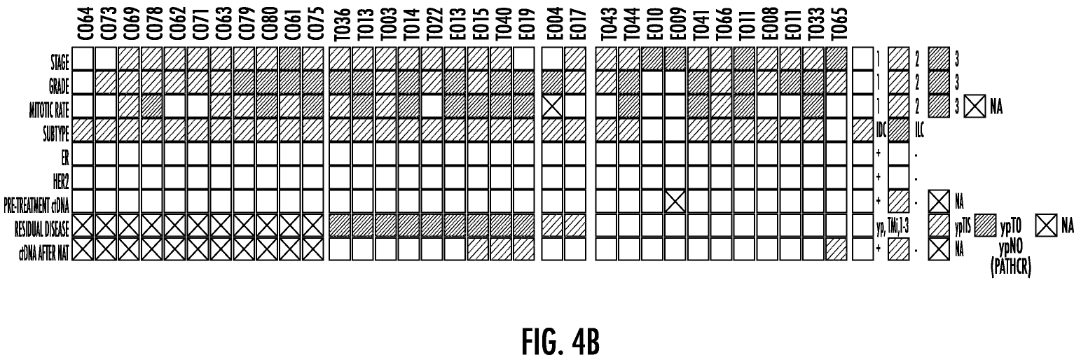

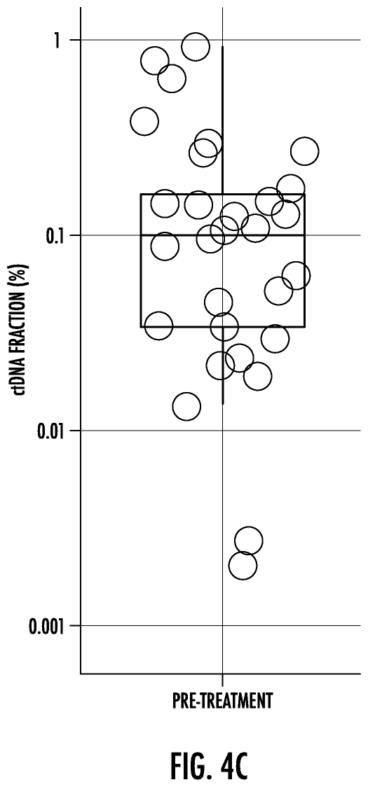

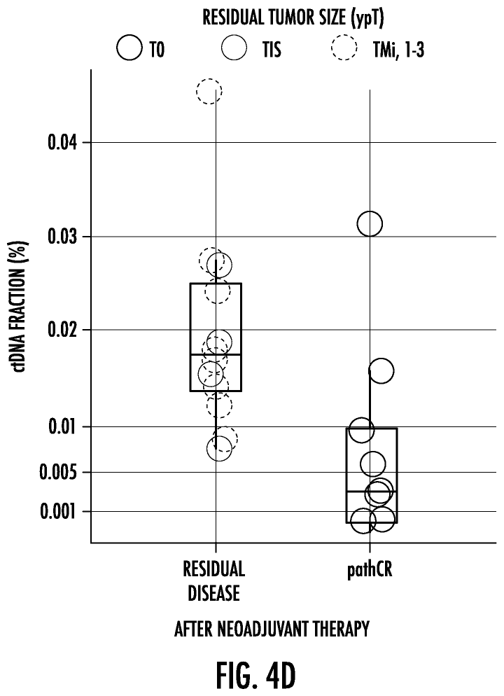

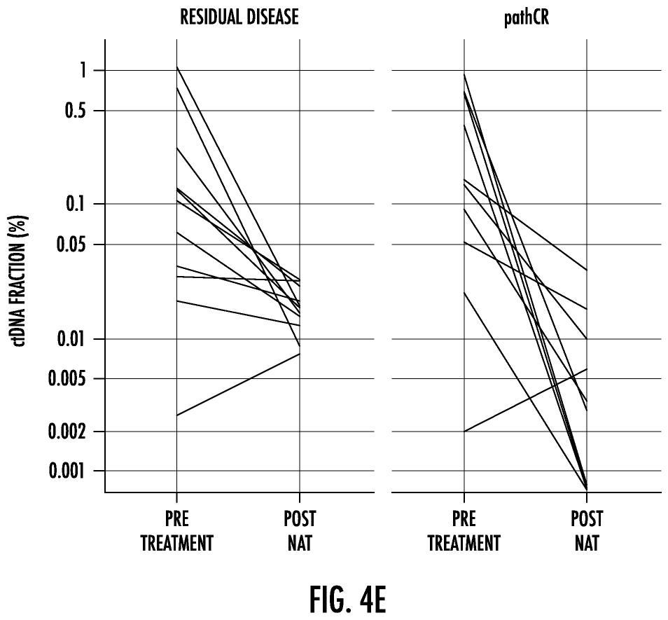

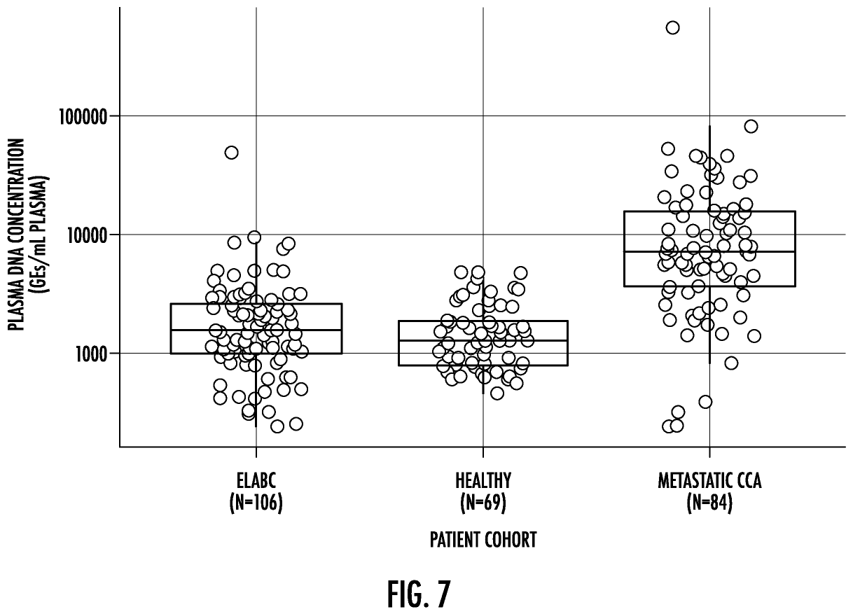

To evaluate whether TARDIS enables residual disease detection in early and locally advanced cancer patients, we analyzed blood samples obtained from 33 patients with Stage I-III breast cancer, of whom 22 patients were treated with neoadjuvant therapy. Distributions of key clinical characteristics of the cohort are presented in FIG. 4A. Most patients presented with Stage II disease (24/33 patients) and invasive ductal carcinoma (30/33 patients). The majority of patients were diagnosed with ER+ HER2− cancer (17/33 patients). 7/33 and 9/33 patients had HER2+ cancer and TNBC respectively. We performed whole exome sequencing of DNA from diagnostic tumor biopsies and matched germline samples, achieving 177× and 143× mean coverage respectively (Table 4). Mean tumor cellularity inferred from exome sequencing analysis was 41% (range 14%-85%). A mean of 700 somatic mutations (range 147-4904) were called across tumor samples and we identified a mean of 65.8 putative founder mutations per patient (range 9-286). By using an aggressive filtering strategy and after further excluding primers amplifying erroneously in validation experiments with control samples, we analyzed 80 serial plasma samples obtained from 33 patients (1-4 samples per patient, Data S3) for 6-115 mutations per patient (mean 30 and median 18 mutations). Across this cohort, validated TARDIS assays covered a mean of 55% putative founder mutations found in the corresponding tumor sample (range 5%-90%, median 67%). Samples were collected prior to commencement of therapy, during NAT and after completion of NAT before surgery. Input plasma DNA amounts were 4.8-34.5 ng per sample (mean 14.1, median 12.7), obtained from 1.2-4.7 mL plasma (mean 3.8, median 4.0) and analyzed in 1-2 replicate TARDIS experiments. Total plasma DNA concentration was a median of 5.3 ng/mL plasma (range 0.83-165.1, mean 8.5). The distribution of total plasma DNA concentration was indistinguishable from healthy volunteers and significantly lower than patients with metastatic cancer (Wilcoxon rank sum test p=0.0789 and p=2.2×10−16, FIG. 7).

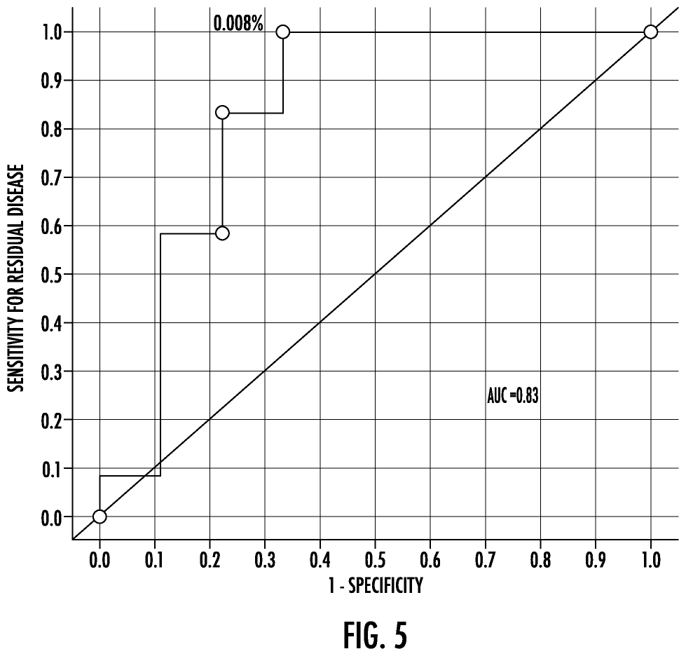

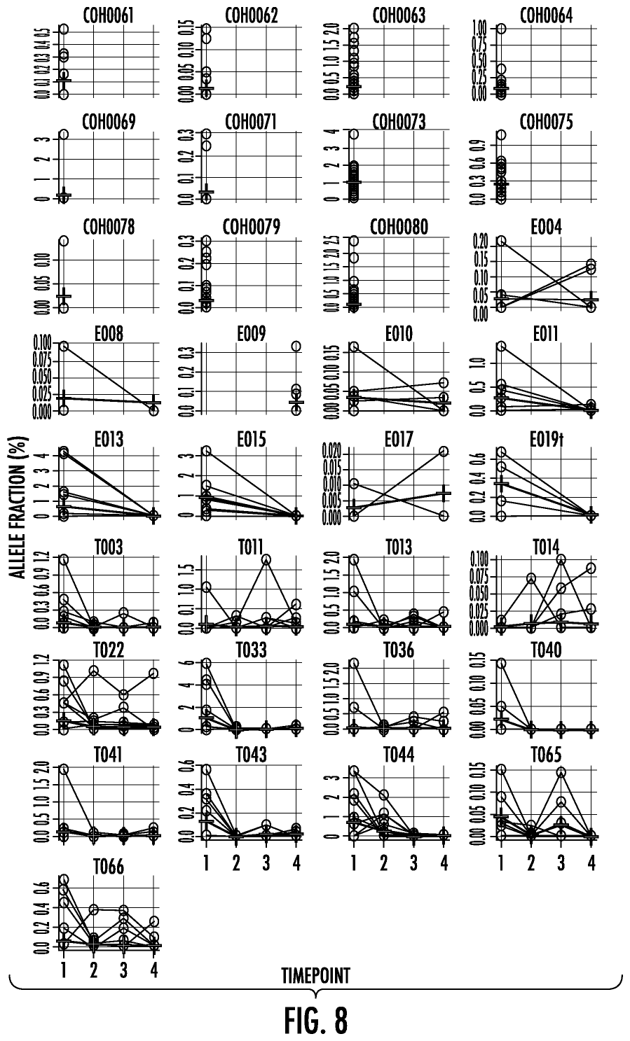

Prior to treatment, we detected ctDNA in 32/32 patients at tumor fractions of 0.002%-1.06% (mean 0.23%, median 0.11%), supported by 2-53 distinct mutation events (mean 10.2, median 7.0) and 3-1638 mutant RFs (mean 217.5, median 54.5, Data S4). To ensure analysis of multiple mutations did not increase false positives, we performed multiple-testing correction (Bonferroni correction) and required a combined p<0.05 for each sample. Baseline plasma sequencing failed in one patient (E009). Plasma samples after completion of neoadjuvant therapy were analyzed in 22 patients. ctDNA was detected in 17/22 patients including 12/13 patients with invasive or in situ residual disease and 5/9 patients with pathCR (no evidence of tumor cells in the resected tissue). In one patient with invasive residual disease (T065), ctDNA was undetectable in the last blood sample after completion of NAT, likely due to a combination of limited plasma DNA available for analysis (8.7 ng compared to mean 16.8 ng for samples obtained after NAT) and limited number of targets analyzed (11 compared to mean of 30 across the entire cohort). We calculated the theoretical maximum number of molecules analyzed for each sample (the product of input haploid genome copies and number of mutations targeted). For patient T065, maximum number of analyzed molecules in the plasma DNA sample after completion of therapy was 26,272, the lowest among the post-NAT samples, compared to mean of 64,375 molecules per sample. We excluded T065 from further analysis of samples after completion of NAT. In patients with detectable ctDNA after NAT, tumor fraction was 0.003%-0.045% (mean 0.018%, median 0.016%), supported by 1-7 distinct mutation events (mean 3.6, median 4.0) and 2-82 mutant RFs (mean 18.8, median 13). Median ctDNA levels after completion of NAT were 5.7 fold lower in patients who achieved pathCR compared to patients with residual disease (median AFs 0.003% vs. 0.018% respectively, Wilcoxon Rank Sum one-sided p=0.0057, FIG. 4D). We observed a decrease in ctDNA levels after NAT compared to pre-treatment levels in all but 2 patients (median decrease of 84%, Wilcoxon signed rank paired one-sided p=9.5×10-6, FIG. 4E). In patients who achieved pathCR, median decrease in ctDNA levels was 96% while in patients with residual disease observed at surgery, median decrease was 77% (Wilcoxon rank-sum one-sided p=0.055). Temporal changes in variant AFs for multiple mutations within each patient agreed with each other, unless affected by sampling variation as ctDNA levels decreased during treatment (FIG. 8). Using ctDNA levels after NAT to differentiate the two groups, we achieved an AUC of 0.83 (n=21 patients, FIG. 5). The AUC improved to 1 and 0.89 when patients were analyzed as TNBC and ER+ breast cancer sub-groups respectively (n=9 and 11 patients).

### Discussion

Patients with early and locally advanced cancers are increasingly treated with neoadjuvant systemic therapy to downstage their tumors and improve outcomes of localized treatment such as surgical resection and radiation therapy. Across some cancer subtypes such as breast, rectal and esophageal cancers, 20%-30% patients achieve pathological Complete Response following neoadjuvant therapy i.e. no evidence of tumor cells is found in surgically resected tissue (2, 15, 16). Achieving pathCR is a biomarker for good prognosis but histopathological evaluation of surgically resected tissue remains the only reliable method to establish pathCR. Imaging and clinical assessment of response have been unable to predict pathCR with high accuracy and no circulating biomarkers have been informative in this setting (4, 5). Our results reveal that ctDNA levels after completion of neoadjuvant therapy for breast cancer are significantly higher in patients with residual disease at the time of surgery compared to patients with pathCR.

Several earlier studies have evaluated whether ctDNA analysis can be informative of response to neoadjuvant therapy in breast cancer. However, these studies were limited in technical sensitivity and precision for ctDNA analysis because ctDNA levels in non-metastatic cancer patients are extremely low, even prior to treatment. In our study, ctDNA was detected in 100% of early and locally advanced breast cancer patients before treatment (95% CI 89%-100%), improving on earlier reports of 50%-75% ctDNA detection at baseline (7, 17). High sensitivity for ctDNA detection prior to treatment is a pre-requisite for any approach used for residual disease testing, considering tumor burden is generally higher at presentation, when tumors are observed clinically and on imaging studies. Median pre-treatment ctDNA level in our study was 0.11%, about 25-100 times lower than ctDNA levels reported in metastatic breast cancer patients (13, 18). After completion of neoadjuvant therapy, we observed a significant difference in ctDNA levels between patients with residual disease and those who achieved pathological Complete Response. To our best assessment of current literature, such a difference has not been reported previously and most studies find ctDNA levels become undetectable in >90% of patients after neoadjuvant therapy regardless of residual disease status (10-12, 19). In our study, after completion of neoadjuvant therapy, median ctDNA levels were 0.017% and 0.003% in patients with residual disease and pathCR respectively. These levels are below the limit of detection of most current and reported ctDNA analysis methods.

To achieve sensitivity and quantitative precision required for ctDNA analysis in non-metastatic cancer patients, we have developed a novel method for tumor-guided ctDNA analysis that leverages multiple mutations together with improvements in sequencing library preparation and informatics analysis. Earlier studies investigating ctDNA quantification for longitudinal treatment monitoring have targeted single recurrent or patient-specific mutations for plasma DNA analysis using digital PCR or digital sequencing (8, 18). While these studies have been informative of large changes in ctDNA levels when patients respond to treatment, ctDNA typically becomes undetectable in mid-treatment samples, even in metastatic cancer patients who have clearly measurable stable disease on imaging (18). For detection of residual disease in non-metastatic cancer patients, the challenges of ctDNA analysis include limited clinically accessible volumes of blood, low concentrations of plasma DNA and loss of input DNA material during analysis. To overcome these challenges and improve sensitivity, several groups are developing new strategies to sample the plasma DNA genome at multiple loci simultaneously. One approach is to analyze multiple genomic regions using targeted sequencing of recurrent cancer genes with high sequencing coverage and to integrate results from multiple mutations in each patient (6). However, such approaches typically do not yield more than 2-4 mutations per patient, limiting the maximum sensitivity achieved regardless of depth of sequencing. More recently, analysis of multiple patient-specific mutations pre-identified in the tumor tissue has emerged as an attractive alternative including PCR-based sequencing of dozens of mutations, hybrid-capture enrichment of sequencing libraries for dozens to thousands of mutations and whole genome sequencing. At various stages of development, these approaches generally improve on the current limit of detection for ctDNA analysis (˜0.1% mutation allele fraction) but each approach has some limitations.

Conventional multiplexed PCR-based approaches have been limited by the high background error rates observed (9). An alternative is to incorporate UMIs during the first few cycles of PCR to overcome background errors but this limits molecular conversion because template DNA molecules not incorporated within the first 2-3 cycles are excluded from further analysis (20-22). In addition, it limits multiplexing capacity and requires optimization for patient-specific assays. In contrast, ligation-based sequencing library preparation enables a wider analysis of the genome but this has limited molecular conversion and loses up to 90% of template DNA molecules due to inefficient ligation (23, 24). Personalized hybrid capture enrichment can overcome this limitation by incorporating thousands of mutations but such a higher number of mutations are only accessible in a few tumor types and identifying them requires whole genome sequencing analysis of high cellularity tumor samples and corresponding normal tissue. Together with synthesis of customized hybrid capture biotinylated baits for each patient, this approach is currently very expensive. An even wider look can be achieved by whole genome sequencing of plasma DNA, either by direct counting of mutated DNA molecules across the genome or integration of genome-wide patient-specific mutational signatures. However, both approaches will require WGS of high-cellularity tumor tissue upfront and WGS of plasma DNA at the required depth of coverage remains prohibitively expensive for a repeatable longitudinal ctDNA test.

In contrast to on-going efforts highlighted above, TARDIS combines the strengths of PCR-based methods (such as minimizing losses of template DNA molecules) and ligation-based methods (such as incorporation of UMIs, preservation of fragment sizes and hundreds-fold multiplexing). This combination achieves a balance between depth and breadth of tumor genome analyzed, investigating dozens to hundreds of patient-specific mutations with deep coverage. TARDIS relies on exome-wide tumor analysis which is clinically much more feasible in the foreseeable future than tumor whole genome sequencing. In addition, it generates greater depth of sequencing coverage and enables more confident identification of putative founder mutations even in lower cellularity tumors. In order to aggregate multiple patient-specific mutations to improve detection sensitivity and quantitative precision for ctDNA analysis, targeted mutations are assumed to be equally informative i.e. they are founder mutations and shared by all tumor cells. Sub-clonal mutations are more likely to be lost due to population bottlenecks during treatment and become uninformative for residual disease detection (9, 14). Using a combination of founder and subclonal mutations may lower the real world sensitivity of the assay, although tumor specificity will remain unaffected. Similarly, an aggregate ctDNA fraction calculated using a mix of founder and sub-clonal mutations may not reflect true tumor burden and can complicate both, assessment of longitudinal changes in ctDNA levels within a patient's clinical course and comparison of ctDNA levels across a cohort of patients due to varying contributions of founder and subclonal mutations. Definitive identification of founder mutations requires multisite sequencing but obtaining multiple biopsies remains clinically challenging. In the current study, we have combined two informatics approaches to maximize the fraction of target mutations likely to be founder.

TARDIS assays require design, synthesis and empirical validation of patient-specific primer panels. However, we have streamlined and automated the design process to successfully target 55% of putative founder mutations per patient on average. Unlike biotinylated oligonucleotides for enrichment by hybridization, we rely on conventional primer synthesis and require a limited sequencing footprint, making our approach more cost-effective and enabling more frequent and longitudinal analysis of plasma samples. The initial cost and turn-around time required for developing patient-specific assays includes exome sequencing of tumor DNA from diagnostic tumor biopsies and germline DNA from peripheral blood leukocytes, routinely performed within 2 weeks of receiving a tumor specimen at our institution. Using automated informatics pipelines, a TARDIS assay can be designed, synthesized and empirically validated for each patient within 1-2 weeks thereafter. Hence, the total turnaround time for development of a patient-specific assay is 3-4 weeks after a diagnostic biopsy, well within the timeframe required for clinical decision making for neoadjuvantly treated cancer patients.

We also report extensive evaluation of analytical performance using commercially available reference samples. Sequencing library preparation typically loses the large majority of input DNA material during early steps such as ligation of adapters. This is particularly challenging for ctDNA analysis because limited blood volumes can be accessed clinically and plasma DNA concentrations are low. We tried to overcome this challenge, while keeping any polymerase-induced errors in check, by using linear pre- amplification of input DNA. To measure our efficiency of molecular conversion, we used a unique approach based on sensitivity and reproducibility across dozens of replicates of known reference samples. We compared observed sensitivity and precision at tumor allele fractions as low as 3 in 10⁵ with expected sensitivity and precision based on Poisson distribution and inferred effective conversion efficiency of 26%-39%. This approach is in contrast with earlier reports relying on unique molecular coverage in the targeted region, a metric that is susceptible to molecular and informatics artifacts due to sequencing and polymerase-induced errors within unique molecular identifiers and tag-switching (polymerase-induced recombination of UMIs). We propose benchmarking of current and future methods for ctDNA analysis using a similar approach, which can also be applied to non-UMI based methods such as conventional amplicon sequencing.

Our results demonstrate potential applications of a novel ctDNA analysis approach for monitoring response in neoadjuvantly treated cancer patients. We have shown that ctDNA levels after completion of neoadjuvant therapy can predict pathological Complete Response in early and locally advanced breast cancer. Together with imaging and clinical assessment, ctDNA levels could inform the choice and extent of local treatment such as surgical resection or radiation. The threshold for ctDNA levels predictive of residual disease will likely vary within clinical subtypes of breast cancer and across other cancer types. Larger clinical studies are planned and on-going to validate our findings and to refine clinically relevant diagnostic thresholds. We have also observed a decrease in ctDNA levels during neoadjuvant therapy which is greater in magnitude when patients achieve pathCR. This highlights the utility of improved quantitative precision achieved using a multi-mutation assay and suggests future studies could evaluate whether the magnitude of early decrease in ctDNA levels during neoadjuvant treatment is informative of therapeutic benefit, enabling adaptive treatment designs to rapidly identify systemic treatment options that work for individual patients. Overall, ctDNA analysis using sensitive and accurate approaches such as TARDIS can enable development of clinical strategies for individualized management of patients treated with curative intent.

We did not detect any ctDNA in one patient with residual disease after completion of neoadjuvant therapy, most likely due to a combination of low plasma DNA concentration and a limited number of mutations assayed for this patient. ctDNA was detected in the same patient in 2 plasma samples collected 6 weeks and 12 weeks earlier, suggesting potential approaches to overcome this limitation in future clinical studies including targeting a greater number of putative founder mutations and analyzing larger blood volumes. Although in the current study, we analyzed up to 4 mL plasma obtained from 10 mL blood samples, it is conceivable to collect up to 30 mL blood at a single time point. It is also feasible to collect and analyze plasma samples over multiple days after completion of therapy. If ctDNA is cleared from blood and remains undetectable in multiple consecutive samples, this could accurately rule out residual disease.

Overtreatment of early stage cancer patients remains a challenge in cancer medicine, likely to become more relevant as newer blood- and imaging-based early detection approaches gain credence (25). Most efforts to optimize treatments have focused on tissue-based predictive biomarkers to assess risk of tumor recurrence (26). Our results suggest blood-based residual disease testing during treatment can further help individualize the choice and extent of each treatment modality. Establishing clinical validity and utility for ctDNA monitoring and residual disease detection will require larger and prospective studies with long-term clinical follow-up. Once validated, using residual disease detection to individualize cancer management could substantially reduce treatment-related morbidity while preserving clinical outcomes.

TABLE 4
Tumor and germline sequencing statistics.

| | Tumor | Germline | Tumor | Somatic | Putative | | % |
|---|---|---|---|---|---|---|---|
| | Sequencing | Sequencing | Purity | Mutations | Founder | Mutations | Founders |
| Patient | Coverage | Coverage | (Sequenza) | Called | Mutations | Analyzed | Analyzed |
| C061 | 197 | 127 | 15% | 350 | 17 | 12 | 71% |
| C062 | 223 | 115 | 85% | 437 | 45 | 39 | 87% |
| C063 | 226 | 120 | 67% | 710 | 134 | 99 | 74% |
| C064 | 151 | 132 | 36% | 503 | 33 | 28 | 85% |
| C069 | 210 | 122 | 29% | 333 | 34 | 22 | 65% |
| C071 | 94 | 120 | 22% | 495 | 20 | 17 | 85% |
| C073 | 208 | 127 | 14% | 361 | 36 | 30 | 83% |
| C075 | 232 | 128 | 76% | 475 | 47 | 35 | 74% |
| C078 | 90 | 142 | 33% | 2236 | 9 | 6 | 67% |
| C079 | 250 | 118 | 28% | 571 | 112 | 67 | 60% |
| C080 | 213 | 122 | 39% | 1130 | 140 | 115 | 82% |
| E004 | 141 | 112 | 27% | 1586 | 62 | 10 | 16% |
| E008 | 302 | 196 | 28% | 546 | 30 | 10 | 33% |
| E010 | 358 | 212 | 49% | 425 | 46 | 8 | 17% |
| E011 | 389 | 173 | 39% | 635 | 83 | 12 | 14% |
| E013 | 139 | 134 | 23% | 1128 | 45 | 11 | 24% |
| E015 | 325 | 211 | 53% | 607 | 105 | 9 | 9% |
| E017 | 270 | 261 | 37% | 416 | 104 | 15 | 14% |
| E019 | 135 | 130 | 50% | 1897 | 13 | 9 | 69% |
| E009 | 81 | 137 | 33% | 4904 | 286 | 14 | 5% |
| T003 | 114 | 99 | 53% | 314 | 46 | 30 | 65% |
| T011 | 141 | 151 | 46% | 269 | 77 | 57 | 74% |
| T013 | 93 | 146 | 50% | 371 | 64 | 55 | 86% |
| T014 | 103 | 96 | 26% | 332 | 160 | 23 | 14% |
| T022 | 129 | 161 | 17% | 233 | 44 | 36 | 82% |
| T033 | 118 | 93 | 30% | 322 | 78 | 18 | 23% |
| T036 | 122 | 166 | 22% | 350 | 108 | 89 | 82% |
| T040 | 81 | 70 | 74% | 207 | 29 | 9 | 31% |
| T041 | 263 | 228 | 65% | 149 | 22 | 18 | 82% |
| T043 | 96 | 208 | 31% | 147 | 21 | 13 | 62% |
| T044 | 149 | 98 | 68% | 262 | 28 | 20 | 71% |
| T065 | 88 | 92 | 40% | 212 | 43 | 10 | 23% |
| T066 | 98 | 156 | 38% | 190 | 50 | 45 | 90% |

Unless defined otherwise, all technical and scientific terms herein have the same meaning as commonly understood by one of ordinary skill in the art to which this disclosure belongs. Although any methods and materials, similar or equivalent to those described herein, can be used in the practice or testing of the present disclosure, the preferred methods and materials are described herein. All publications, patents, and patent publications cited are incorporated by reference herein in their entirety for all purposes.

The publications discussed herein are provided solely for their disclosure prior to the filing date of the present application. Nothing herein is to be construed as an admission that the present invention is not entitled to antedate such publication by virtue of prior invention.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

### REFERENCES

1. S. J. Katz, R. Jagsi, M. Morrow, Reducing Overtreatment of Cancer With Precision Medicine: Just What the Doctor Ordered. JAMA 319, 1091-1092 (2018).
2. P. Cortazar, L. Zhang, M. Untch, K. Mehta, J. Costantino, N. Wolmark, H. Bonnefoi, D. Cameron, L. Gianni, P. Valagussa, Meta-analysis results from the collaborative trials in neoadjuvant breast cancer (CTNeoBC). Cancer Res 72, S1-S11 (2012).
3. W. F. Symmans, C. Wei, R. Gould, X. Yu, Y. Zhang, M. Liu, A. Walls, A. Bousamra, M. Ramineni, B. Sinn, K. Hunt, T. A. Buchholz, V. Valero, A. U. Buzdar, W. Yang, A. M. Brewster, S. Moulder, L. Pusztai, C. Hatzis, G. N. Hortobagyi, Long-Term Prognostic Risk After Neoadjuvant Chemotherapy Associated With Residual Cancer Burden and Breast Cancer Subtype. J Clin Oncol 35, 1049-1060 (2017).
4. A. B. Chagpar, L. P. Middleton, A. A. Sahin, P. Dempsey, A. U. Buzdar, A. N. Mirza, F. C. Ames, G. V. Babiera, B. W. Feig, K. K. Hunt, H. M. Kuerer, F. Meric-Bernstam, M. I. Ross, S. E. Singletary, Accuracy of physical examination, ultrasonography, and mammography in predicting residual pathologic tumor size in patients treated with neoadjuvant chemotherapy. Annals of surgery 243, 257-264 (2006).
5. Y. Yuan, X. S. Chen, S. Y. Liu, K. W. Shen, Accuracy of MRI in prediction ofpathologic complete remission in breast cancer after preoperative therapy: a meta-analysis. AJR. American journal of roentgenology 195, 260-268 (2010).
6. A. A. Chaudhuri, J. J. Chabon, A. F. Lovejoy, A. M. Newman, H. Stehr, T. D. Azad, M. S. Khodadoust, M. S. Esfahani, C. L. Liu, L. Zhou, F. Scherer, D. M. Kurtz, C. Say, J. N. Carter, D. J. Merriott, J. C. Dudley, M. S. Binkley, L. Modlin, S. K. Padda, M. F. Gensheimer, R. B. West, J. B. Shrager, J. W. Neal, H. A. Wakelee, B. W. Loo, Jr., A. A. Alizadeh, M. Diehn, Early Detection of Molecular Residual Disease in Localized Lung Cancer by Circulating Tumor DNA Profiling. Cancer Discov 7, 1394-1403 (2017).
7. I. Garcia-Murillas, G. Schiavon, B. Weigelt, C. Ng, S. Hrebien, R. J. Cutts, M. Cheang, P. Osin, A. Nerurkar, I. Kozarewa, J. A. Garrido, M. Dowsett, J. S. Reis-Filho, I. E. Smith, N. C. Turner, Mutation tracking in circulating tumor DNA predicts relapse in early breast cancer. Sci Transl Med 7, 302ra133 (2015).
8. J. Tie, Y. Wang, C. Tomasetti, L. Li, S. Springer, I. Kinde, N. Silliman, M. Tacey, H. L. Wong, M. Christie, S. Kosmider, I. Skinner, R. Wong, M. Steel, B. Tran, J. Desai, I. Jones, A. Haydon, T. Hayes, T. J. Price, R. L. Strausberg, L. A. Diaz, Jr., N. Papadopoulos, K. W. Kinzler, B. Vogelstein, P. Gibbs, Circulating tumor DNA analysis detects minimal residual disease and predicts recurrence in patients with stage II colon cancer. Sci Transl Med 8, 346ra392 (2016).
9. C. Abbosh, N. J. Birkbak, G. A. Wilson, M. Jamal-Hanjani, T. Constantin, R. Salari, J. Le Quesne, D. A. Moore, S. Veeriah, R. Rosenthal, T. Marafioti, E. Kirkizlar, T. B. K. Watkins, N. McGranahan, S. Ward, L. Martinson, J. Riley, F. Fraioli, M. Al Bakir, E. Gronroos, F. Zambrana, R. Endozo, W. L. Bi, F. M. Fennessy, N. Sponer, D. Johnson, J. Laycock, S. Shafi, J. Czyzewska-Khan, A. Rowan, T. Chambers, N. Matthews, S. Turajlic, C. Hiley, S. M. Lee, M. D. Forster, T. Ahmad, M. Falzon, E. Borg, D. Lawrence, M. Hayward, S. Kolvekar, N. Panagiotopoulos, S. M. Janes, R. Thakrar, A. Ahmed, F. Blackhall, Y. Summers, D. Hafez, A. Naik, A. Ganguly, S. Kareht, R. Shah, L. Joseph, A. Marie Quinn, P. A. Crosbie, B. Naidu, G. Middleton, G. Langman, S. Trotter, M. Nicolson, H. Remmen, K. Kerr, M. Chetty, L. Gomersall, D. A. Fennell, A. Nakas, S. Rathinam, G. Anand, S. Khan, P. Russell, V. Ezhil, B. Ismail, M. Irvin-Sellers, V. Prakash, J. F. Lester, M. Kornaszewska, R. Attanoos, H. Adams, H. Davies, D. Oukrif, A. U. Akarca, J. A. Hartley, H. L. Lowe, S. Lock, N. Iles, H. Bell, Y. Ngai, G. Elgar, Z. Szallasi, R. F. Schwarz, J. Herrero, A. Stewart, S. A. Quezada, K. S. Peggs, P. Van Loo, C. Dive, C. J. Lin, M. Rabinowitz, H. Aerts, A. Hackshaw, J. A. Shaw, B. G. Zimmermann, T. R. consortium, P. consortium, C. Swanton, Phylogenetic ctDNA analysis depicts early-stage lung cancer evolution. Nature 545, 446-451 (2017).
10. F. Rothe, M. J. Silva, D. Venet, C. Campbell, I. Bradbury, G. Rouas, E. de Azambuja, M. Maetens, D. Fumagalli, V. Rodrik-Outmezguine, S. Di Cosimo, D. D. Rosa, S. K. Chia, A. M. Wardley, T. Ueno, W. Janni, J. Huober, J. Baselga, M. Piccart, S. Loi, C. Sotiriou, S. J. Dawson, M. Ignatiadis, Circulating tumor DNA in HER2 amplified breast cancer: a translational research substudy of the NeoALTTO phase 3 trial. Clinical cancer research: an official journal of the American Association for Cancer Research, (2019).
11. J. Tie, J. D. Cohen, Y. Wang, L. Li, M. Christie, K. Simons, H. Elsaleh, S. Kosmider, R. Wong, D. Yip, M. Lee, B. Tran, D. Rangiah, M. Burge, D. Goldstein, M. Singh, I. Skinner, I. Faragher, M. Croxford, C. Bampton, A. Haydon, I. T. Jones, S. K. C, T. Price, M. J. Schaefer, J. Ptak, L. Dobbyn, N. Silliman, I. Kinde, C. Tomasetti, N. Papadopoulos, K. Kinzler, B. Volgestein, P. Gibbs, Serial circulating tumour DNA analysis during multimodality treatment of locally advanced rectal cancer: a prospective biomarker study. Gut, (2018).
12. F. Riva, F. C. Bidard, A. Houy, A. Saliou, J. Madic, A. Rampanou, C. Hego, M. Milder, P. Cottu, M. P. Sablin, A. Vincent-Salomon, O. Lantz, M. H. Stern, C. Proudhon, J. Y. Pierga, Patient-Specific Circulating Tumor DNA Detection during Neoadjuvant Chemotherapy in Triple-Negative Breast Cancer. Clin Chem 63, 691-699 (2017).
13. D. G. Stover, H. A. Parsons, G. Ha, S. S. Freeman, W. T. Barry, H. Guo, A. D. Choudhury, G. Gydush, S. C. Reed, J. Rhoades, D. Rotem, M. E. Hughes, D. A. Dillon, A. H. Partridge, N. Wagle, I. E. Krop, G. Getz, T. R. Golub, J. C. Love, E. P. Winer, S. M. Tolaney, N. U. Lin, V. A. Adalsteinsson, Association of Cell-Free DNA Tumor Fraction and Somatic Copy Number Alterations With Survival in Metastatic Triple- Negative Breast Cancer. J Clin Oncol 36, 543-553 (2018).
14. M. Murtaza, S. J. Dawson, K. Pogrebniak, O. M. Rueda, E. Provenzano, J. Grant, S. F. Chin, D. W. Tsui, F. Marass, D. Gale, H. R. Ali, P. Shah, T. Contente-Cuomo, H. Farahani, K. Shumansky, Z. Kingsbury, S. Humphray, D. Bentley, S. P. Shah, M. Wallis, N. Rosenfeld, C. Caldas, Multifocal clonal evolution characterized using circulating tumour DNA in a case of metastatic breast cancer. Nat Commun 6, 8760 (2015).
15. M. Blum Murphy, L. Xiao, V. R. Patel, D. M. Maru, A. M. Correa, G. A. F, Z. Liao, R. Komaki, S. H. Lin, H. D. Skinner, A. Vaporciyan, G. L. Walsh, S. G. Swisher, B. Sepesi, J. H. Lee, M. S. Bhutani, B. Weston, W. L. Hofstetter, J. A. Ajani, Pathological complete response in patients with esophageal cancer after the trimodality approach: The association with baseline variables and survival-The University of Texas MD Anderson Cancer Center experience. Cancer 123, 4106-4113 (2017).
16. A. Cercek, K. A. Goodman, C. Hajj, E. Weisberger, N. H. Segal, D. L. Reidy-Lagunes, Z. K. Stadler, A. J. Wu, M. R. Weiser, P. B. Paty, J. G. Guillem, G. M. Nash, L. K. Temple, J. Garcia-Aguilar, L. B. Saltz, Neoadjuvant chemotherapy first, followed by chemoradiation and then surgery, in the management of locally advanced rectal cancer. J Natl Compr Canc Netw 12, 513-519 (2014).
17. C. Bettegowda, M. Sausen, R. J. Leary, I. Kinde, Y. Wang, N. Agrawal, B. R. Bartlett, H. Wang, B. Luber, R. M. Alani, E. S. Antonarakis, N. S. Azad, A. Bardelli, H. Brem, J. L. Cameron, C. C. Lee, L. A. Fecher, G. L. Gallia, P. Gibbs, D. Le, R. L. Giuntoli, M. Goggins, M. D. Hogarty, M. Holdhoff, S. M. Hong, Y. Jiao, H. H. Juhl, J. J. Kim, G. Siravegna, D. A. Laheru, C. Lauricella, M. Lim, E. J. Lipson, S. K. Marie, G. J. Netto, K. S. Oliner, A. Olivi, L. Olsson, G. J. Riggins, A. Sartore-Bianchi, K. Schmidt, M. Shih l, S. M. Oba-Shinjo, S. Siena, D. Theodorescu, J. Tie, T. T. Harkins, S. Veronese, T. L. Wang, J. D. Weingart, C. L. Wolfgang, L. D. Wood, D. Xing, R. H. Hruban, J. Wu, P. J. Allen, C. M. Schmidt, M. A. Choti, V. E. Velculescu, K. W. Kinzler, B. Vogelstein, N. Papadopoulos, L. A. Diaz, Jr., Detection of circulating tumor DNA in early- and late- stage human malignancies. Sci Transl Med 6, 224ra224 (2014).
18. S. J. Dawson, D. W. Tsui, M. Murtaza, H. Biggs, O. M. Rueda, S. F. Chin, M. J. Dunning, D. Gale, T. Forshew, B. Mahler-Araujo, S. Rajan, S. Humphray, J. Becq, D. Halsall, M. Wallis, D. Bentley, C. Caldas, N. Rosenfeld, Analysis of circulating tumor DNA to monitor metastatic breast cancer. N Engl J Med 368, 1199-1209 (2013).
19. T. M. Butler, C. T. Boniface, K. Johnson-Camacho, S. Tabatabaei, D. Melendez, T. Kelley, J. Gray, C. L. Corless, P. T. Spellman, Circulating tumor DNA dynamics using patient-customized assays are associated with outcome in neoadjuvantly treated breast cancer. Cold Spring Harb Mol Case Stud 5, (2019).
20. A. Stahlberg, P. M. Krzyzanowski, J. B. Jackson, M. Egyud, L. Stein, T. E. Godfrey, Simple, multiplexed, PCR-based barcoding of DNA enables sensitive mutation detection in liquid biopsies using sequencing. Nucleic Acids Res 44, e105 (2016).
21. I. Kinde, J. Wu, N. Papadopoulos, K. W. Kinzler, B. Vogelstein, Detection and quantification of rare mutations with massively parallel sequencing. Proceedings of the National Academy of Sciences of the United States of America 108, 9530-9535 (2011).
22. A. Narayan, N. J. Carriero, S. N. Gettinger, J. Kluytenaar, K. R. Kozak, T. I. Yock, N. E. Muscato, P. Ugarelli, R. H. Decker, A. A. Patel, Ultrasensitive measurement of hotspot mutations in tumor DNA in blood using error-suppressed multiplexed deep sequencing. Cancer Res 72, 3492-3498 (2012).
23. A. Zviran, S. T. Hill, R. Schulman, M. Shah, S. Deochand, G. Ha, S. Reed, D. Rotem, G. Gydush, J. Rhoades, K. Huang, W. Liao, D. Maloney, N. Omans, M. Malbari, C. F. Spinelli, S. Kazancioglu, N. Robine, V. Adalsteinsson, B. Houck-Loomis, N. Altorki, D. A. Landau, Abstract 3247: Genome-wide cell-free DNA mutation integration for sensitive cancer detection. Cancer Research 78, 3247-3247 (2018).
24. A. Ruiz-Valdepenas, K. Heider, G. Doughton, W. Qian, C. Massie, D. Chandrananda, C. Smith, D. Gale, E. Moseley, C. Castedo, A. Stone, C. Thorbinson, T. Eisen, D. Rassl, S. Harden, R. Rintoul, N. Rosenfeld, M A 11.02 Circulating Tumor DNA in Early Stage NSCLC: High Sensitivity Analysis in Low Burden Disease. LUCID Study Update. Journal of Thoracic Oncology 12, S1843-S1844 (2017).
25. J. D. Cohen, L. Li, Y. Wang, C. Thoburn, B. Afsari, L. Danilova, C. Douville, A. A. Javed, F. Wong, A. Mattox, R. H. Hruban, C. L. Wolfgang, M. G. Goggins, M. Dal Molin, T. L. Wang, R. Roden, A. P. Klein, J. Ptak, L. Dobbyn, J. Schaefer, N. Silliman, M. Popoli, J. T. Vogelstein, J. D. Browne, R. E. Schoen, R. E. Brand, J. Tie, P. Gibbs, H. L. Wong, A. S. Mansfield, J. Jen, S. M. Hanash, M. Falconi, P. J. Allen, S. Zhou, C. Bettegowda, L. A. Diaz, Jr., C. Tomasetti, K. W. Kinzler, B. Vogelstein, A. M. Lennon, N. Papadopoulos, Detection and localization of surgically resectable cancers with a multi- analyte blood test. Science 359, 926-930 (2018).
26. J. A. Sparano, R. J. Gray, D. F. Makower, K. I. Pritchard, K. S. Albain, D. F. Hayes, C. E. Geyer, Jr., E. C. Dees, M. P. Goetz, J. A. Olson, Jr., T. Lively, S. S. Badve, T. J. Saphner, L. I. Wagner, T. J. Whelan, M. J. Ellis, S. Paik, W. C. Wood, P. M. Ravdin, M. M. Keane, H. L. Gomez Moreno, P. S. Reddy, T. F. Goggins, I. A. Mayer, A. M. Brufsky, D. L. Toppmeyer, V. G. Kaklamani, J. L. Berenberg, J. Abrams, G. W. Sledge, Jr., Adjuvant Chemotherapy Guided by a 21-Gene Expression Assay in Breast Cancer. N Engl J Med 379, 111-121 (2018).
27. H. Markus, T. Contente-Cuomo, M. Farooq, W. S. Liang, M. J. Borad, S. Sivakumar, S. Gollins, N. L. Tran, H. D. Dhruv, M. E. Berens, A. Bryce, A. Sekulic, A. Ribas, J. M. Trent, P. M. LoRusso, M. Murtaza, Evaluation of pre-analytical factors affecting plasma DNA analysis. Sci Rep 8, 7375 (2018).
28. H. Li, Aligning sequence reads, clone sequences and assembly contigs with BWA-MEM. arXiv preprint arXiv: 1303.3997, (2013).
29. G. A. Van der Auwera, M. O. Carneiro, C. Hartl, R. Poplin, G. Del Angel, A. Levy-Moonshine, T. Jordan, K. Shakir, D. Roazen, J. Thibault, E. Banks, K. V. Garimella, D. Altshuler, S. Gabriel, M. A. DePristo, From FastQ data to high confidence variant calls: the Genome Analysis Toolkit best practices pipeline. Curr Protoc Bioinformatics 43, 11 10 11-33 (2013).
30. E. Garrison, G. Marth, Haplotype-based variant detection from short-read sequencing. ArXiv e-prints. 2012.
31. K. Cibulskis, M. S. Lawrence, S. L. Carter, A. Sivachenko, D. Jaffe, C. Sougnez, S. Gabriel, M. Meyerson, E. S. Lander, G. Getz, Sensitive detection of somatic point mutations in impure and heterogeneous cancer samples. Nat Biotechnol 31, 213-219 (2013).
32. A. Christoforides, J. D. Carpten, G. J. Weiss, M. J. Demeure, D. D. Von Hoff, D. W. Craig, Identification of somatic mutations in cancer through Bayesian-based analysis of sequenced genome pairs. BMC Genomics 14, 302 (2013).
33. C. T. Saunders, W. S. Wong, S. Swamy, J. Becq, L. J. Murray, R. K. Cheetham, Strelka: accurate somatic small-variant calling from sequenced tumor-normal sample pairs. Bioinformatics 28, 1811-1817 (2012).
34. F. Favero, T. Joshi, A. M. Marquard, N. J. Birkbak, M. Krzystanek, Q. Li, Z. Szallasi, A. C. Eklund, Sequenza: allele-specific copy number and mutation profiles from tumor sequencing data. Ann Oncol 26, 64-70 (2015).
35. N. McGranahan, F. Favero, E. C. de Bruin, N. J. Birkbak, Z. Szallasi, C. Swanton, Clonal status of actionable driver events and the timing of mutational processes in cancer evolution. Sci Transl Med 7, 283ra254 (2015).
36. A. Roth, J. Khattra, D. Yap, A. Wan, E. Laks, J. Biele, G. Ha, S. Aparicio, A. Bouchard-Cote, S. P. Shah, PyClone: statistical inference of clonal population structure in cancer. Nat Methods 11, 396-398 (2014).
37. A. Untergasser, I. Cutcutache, T. Koressaar, J. Ye, B. C. Faircloth, M. Remm, S. G. Rozen, Primer3—new capabilities and interfaces. Nucleic Acids Res 40, el15 (2012).
38. S. M. Kielbasa, R. Wan, K. Sato, P. Horton, M. C. Frith, Adaptive seeds tame genomic sequence comparison. Genome Res 21, 487-493 (2011).
39. C. K. Kwok, Y. Ding, M. E. Sherlock, S. M. Assmann, P. C. Bevilacqua, A hybridization-based approach for quantitative and low-bias single-stranded DNA ligation. Anal Biochem 435, 181-186 (2013).
40. T. Smith, A. Heger, I. Sudbery, UMI-tools: modeling sequencing errors in Unique Molecular Identifiers to improve quantification accuracy. Genome Res 27, 491-499 (2017).

1. A method of treating a cancer in a patient who has undergone a first anti-cancer therapy, the method comprising:
a) obtaining double-stranded cell-free DNA (cfDNA) from a blood sample from the patient;
b) linearly amplifying the cfDNA with target-specific primers to generate single-stranded DNA amplicons, wherein the target-specific primers are generated from a genetic profile of the patient;
c) ligating an adapter oligonucleotide to the 3′-ends of the single-stranded DNA amplicons,
d) performing multiplexed, exponential amplification with target-specific primers and nested primers on the single-stranded DNA amplicons to produce parent polynucleotides;
e) amplifying the parent polynucleotides to produce progeny polynucleotides with associated sample barcodes;
f) sequencing a portion of the progeny polynucleotides to produce sequencing reads of the progeny polynucleotides with associated sample barcodes;
f) aligning mappable portions of the sequencing reads to a human reference genome;
g) grouping a plurality of the sequencing reads into clusters based on the sequence information of the sample barcodes and the beginning and end base positions of the mapped portion of the progeny polynucleotides;
h) detecting, from among a plurality of the clusters, the presence or absence of one or more somatic genetic variants characteristic of the cancer, wherein the presence of the one or more somatic genetic variants or the aggregate quantification of somatic genetic variants surpassing a threshold, indicates cancer persistence and/or recurrence; and
i) administering a second anti-cancer therapy to the patient once cancer recurrence is detected.

2. A method of detecting the presence or absence of one or more somatic genetic variants characteristic of a cancer in a patient, the method comprising:
a) extracting double-stranded cell-free DNA (cfDNA) from a blood sample from the patient;
b) linearly amplifying the cfDNA with target-specific primers to generate single-stranded DNA amplicons, wherein the target-specific primers are generated from a genetic profile of the patient;
c) ligating an adapter oligonucleotide to the 3′-ends of the single-stranded DNA amplicons,
d) performing multiplexed, exponential amplification with target-specific primers and nested primers on the single-stranded DNA amplicons to produce parent polynucleotides;
e) amplifying the parent polynucleotides to produce progeny polynucleotides with associated sample barcodes;
f) sequencing a portion of the progeny polynucleotides to produce sequencing reads of the progeny polynucleotides with associated sample barcodes;
f) aligning mappable portions of the sequencing reads to a human reference genome;
g) grouping a plurality of the sequencing reads into clusters based on the sequence information of the sample barcodes and the beginning and end base positions of the mapped portion of the progeny polynucleotides; and
h) detecting, from among a plurality of the clusters, the presence or absence of one or more somatic genetic variants characteristic of the cancer, wherein the presence of the one or more somatic genetic variants or the aggregate quantification of somatic genetic variants surpassing a threshold indicates cancer recurrence and/or a need for adjustment in cancer treatment.

3. The method of claim 2, further comprising generating a report that includes a cell-free tumor mutation profile of the patient based on the detection of the presence or absence of the one or more somatic genetic variants.

4. The method of claim 3, wherein the report further includes a treatment recommendation for the patient based on the cell-free tumor mutation profile.

5. The method of claim 2, wherein the patient has early stage cancer and the blood sample comprises less than 5 ng cfDNA/mL, less than 4 ng cfDNA/mL, less than 3 ng cfDNA/mL, less than 2 ng cfDNA/mL, or less than 1 ng cfDNA/mL.

6. The method of claim 2, wherein the genetic profile comprises patient-specific putative founder mutations identified with whole genome or whole exome sequencing of tumor biopsy DNA and germline DNA from the patient.

7. The method of claim 2, wherein the one or more somatic genetic variants comprises a single nucleotide variant (SNV), a copy number variation (CNV), an insertion or deletion (indel), a gene fusion, or any combination thereof.

8-10. (canceled)

11. The method of claim 2, wherein the adapter oligonucleotide comprises:
a stem-loop intramolecular nucleotide base pairing;
a hydroxyl group at the 3′-end;
a phosphate at the 5′-end;
a random region complementary to the nucleic acid sequence; and
a random region in the loop comprising a unique molecular identifier (UMI).

12. (canceled)

13. The method of claim 2, further comprising differentiating true low-abundance somatic genetic variants from nucleotide misincorporations that occur during amplification or from nucleotide misreads that occur during sequencing by:
grouping sequencing reads based on fragment size and UMI into read families (RFs);
requiring consensus among all sequencing reads in a read family (RF); and
requiring that a true low-abundance somatic genetic variant be supported by at least two independent RFs of different fragment size.

14. The method of claim 13, wherein the allele fraction (AF) for a somatic genomic variant measured as mutant RFs divided by total RFs is at least 0.5 DNA somatic genomic variant molecules in the amplification reaction.

15. The method of claim 13, wherein the RFs covering each targeted genomic locus are sorted by number of members in the RF and the RFs with the highest number of members up to 5-fold of a known level of input cfDNA in genomic equivalents are selected for detection of somatic genomic variants.

16. The method of claim 15, wherein the cut-off for detection of somatic genomic variants is less than 5-fold or greater than 5-fold.

17. The method of claim 13, further comprising calculating a probability of observing each somatic genetic variant based on a background distribution of mutations in the cfDNA, applying multiple testing correction using the Bonferroni approach, and requiring a corrected p-value of <0.05.

18. The method of claims 13, further comprising assessing probability of error at a locus based on mixed RFs containing multiple members that disagree on nucleotide identity at a target genomic locus.

19. The method of claim 17, wherein background distribution of mutations in cfDNA is calculated using data from adjacent genomic loci.

20. The method of claim 17, wherein the background distribution of mutations in cfDNA is calculated using data from an unrelated set of biological samples not expected to be mutated at the targeted genomic locus or at unrelated genomic loci.

21. The method of claim 2, wherein the target-specific primers simultaneously amplify target regions comprising at least 10 mutations in the cfDNA.

22. The method of claim 2, wherein the target-specific primers amplify target regions comprising a genomic sequence selected from the group consisting of: AKT, GNAQ, GNA11, IDH1, TP53, KRAS, PDGFRA, PIK3CA, APC, EGFR, BRAF, MET, MYC, and RET.

23. (canceled)

24. The method of claim 2, wherein the cancer is selected from the group consisting of: gastrointestinal cancer, prostate cancer, ovarian cancer, breast cancer, head and neck cancer, lung cancer, non-small cell lung cancer, cancer of the nervous system, kidney cancer, retina cancer, skin cancer, liver cancer, pancreatic cancer, genital-urinary cancer, colorectal cancer, renal cancer, hematological cancer, hematological disease, hematological malignancy, minimal residual disease, and bladder cancer.

25. The method of claim 2, wherein a blood sample is collected from the patient for analysis over multiple days after completion of an anti-cancer therapy.

26. The method of claim 2, wherein the patient is a cancer patient that has been treated with a neoadjuvant therapy.


SEQUENCE LISTING (23 sequences)

SEQ ID NO: 1 - DNA, 53 bp, Artificial Sequence
  Hairpin oligonucleotide
  misc_feature (10)..(18): A 9 bp unique molecular identifier/ N= A, T, C, or G.
  misc_feature (36)..(41): N= A, T, C, or G.
  misc_feature (36)..(41): N= A, T, C, or G.
tgctctttgnnnnnnnnngtcagatcggaagagcannnnnnctgcccatagag

SEQ ID NO: 2 - DNA, 17 bp, Artificial Sequence
  Primer
cgctcttccgatctctg

SEQ ID NO: 3 - DNA, 37 bp, Artificial Sequence
  Primer sequence
gtgactggagttcagacgtgtgctcttccgatctgac

SEQ ID NO: 4 - DNA, 67 bp, Artificial Sequence
  Primer sequence
  misc_feature: N= A, T, G, or C
  misc_feature (25)..(30): n is a, c, g, or t
caagcagaagacggcatacgagatnnnnnngtgactggagttcagacgtgtgctcttccgatctgac

SEQ ID NO: 5 - DNA, 61 bp, Artificial Sequence
  Primer sequence
aatgatacggcgaccaccgagatctacactctttccctacacgacgctcttccgatctctg

SEQ ID NO: 6 - DNA, 36 bp, Artificial Sequence
  Primer sequence
acactctttccctacacgacgctcttccgatctctg

SEQ ID NO: 7 - DNA, 37 bp, Artificial Sequence; identical to SEQ ID NO: 3
  Primer sequence

SEQ ID NO: 8 - DNA, 25 bp, Artificial Sequence
  Primer sequence
aggtgccatcattcttgaggaggaa

SEQ ID NO: 9 - DNA, 17 bp, Artificial Sequence
  Primer sequence
ccgccaggtcttgatgt

SEQ ID NO: 10 - DNA, 26 bp, Artificial Sequence
  Primer sequence
tgtcatagggactctggatcccagaa

SEQ ID NO: 11 - DNA, 21 bp, Artificial Sequence
  Primer sequence
agttaaaattcccgtcgctat

SEQ ID NO: 12 - DNA, 20 bp, Artificial Sequence
  Primer sequence
cgtggacaacccccacgtgt

SEQ ID NO: 13 - DNA, 15 bp, Artificial Sequence
  Primer sequence
ccaccgtgcagctca

SEQ ID NO: 14 - DNA, 20 bp, Artificial Sequence
  Primer sequence
tggctgagtcctggcgctgt

SEQ ID NO: 15 - DNA, 19 bp, Artificial Sequence
  Primer sequence
tttcaggatggtggatgtg

SEQ ID NO: 16 - DNA, 22 bp, Artificial Sequence
  Primer sequence
tgcaaaaatatcccccggcttg

SEQ ID NO: 17 - DNA, 22 bp, Artificial Sequence
  Primer sequence
ggatgggtaaaacctatcatca

SEQ ID NO: 18 - DNA, 26 bp, Artificial Sequence
  Primer sequence
gggaagtgaggacgtacactgccttt

SEQ ID NO: 19 - DNA, 21 bp, Artificial Sequence
  Primer sequence
acatagttcgaatcatgcatg

SEQ ID NO: 20 - DNA, 28 bp, Artificial Sequence
  Primer sequence
gctggacaacaaaaatggattggatctt

SEQ ID NO: 21 - DNA, 21 bp, Artificial Sequence
  Primer sequence
cttccacacaattaaacagca

SEQ ID NO: 22 - DNA, 30 bp, Artificial Sequence
  Primer sequence
acacttacacatcactttgcgtggtgtaga

SEQ ID NO: 23 - DNA, 21 bp, Artificial Sequence
  Primer sequence
tgatcaaaaagggattcaatt